(19) United States
(12) Patent Application Publication
(10) Pub. No.: US 2024/0408246 A1
(43) Pub. Date: Dec. 12, 2024

(54) Animal Model With Rapid Onset Of Alzheimer's Amyloid Beta Plaque Pathology

(71) Applicant: Regeneron Pharmaceuticals, Inc., Tarrytown, NY (US)

(72) Inventors: Brian Huang, Basking Ridge, NJ (US); Min Gao, Woodcliff Lake, NJ (US); John Dugan, Hoboken, NJ (US)

(21) Appl. No.: 18/736,791

(22) Filed: Jun. 7, 2024

Related U.S. Application Data
(60) Provisional application No. 63/506,872, filed on Jun. 8, 2023.

(51) Int. Cl. A61K 49/00; A01K 67/0275; C07K 14/47; C12N 9/64; C12N 15/85; C12N 15/86

(52) U.S. Cl. CPC A61K 49/0008; A01K 67/0275; C07K 14/4711; C12N 9/6478; C12N 15/8509; C12N 15/86; C12Y 304/23046; A01K 2217/05; A01K 2227/105; A01K 2267/0312; C12N 2015/8545; C12N 2750/14143

(57) ABSTRACT

Provided herein are compositions comprising a nucleic acid encoding amyloid-beta precursor protein and/or a nucleic acid encoding presenilin-1, cells comprising the compositions, animals comprising the compositions, methods of making the cells and animals, methods of modeling Alzheimer's disease, methods of assessing a therapeutic candidate for the treatment of Alzheimer's disease or amelioration of a symptom or phenotype of Alzheimer's disease, and methods of assessing a therapeutic candidate for the prevention of Alzheimer's disease or prevention of a symptom or phenotype of Alzheimer's disease.

### CROSS-REFERENCE TO RELATED APPLICATIONS

This application claims the benefit of U.S. Application No. 63/506,872, filed Jun. 8, 2023, which is herein incorporated by reference in its entirety for all purposes.

### REFERENCE TO A SEQUENCE LISTING SUBMITTED AS AN XML FILE VIA EFS WEB

The Sequence Listing written in file 614573SEQLIST.xml is 22,759 bytes, was created on Jun. 6, 2024, and is hereby incorporated by reference.

### BACKGROUND

In the Alzheimer's disease field, there are transgenic mouse models that express proteins with Alzheimer's disease (AD) mutations. While these mouse models are extremely useful, they have many disadvantages. First, it is very time-consuming to study how different genes affect AD pathology in transgenic AD models, requiring breeding of these mouse models to other genetic mouse models to generate compound genetic mice. Second, the pathology in transgenic AD models does not appear for an extended period of time. Even in the most aggressive models, most of the pathology does not appear until after six months of age. Better models are needed.

### SUMMARY

Provided herein are compositions comprising a nucleic acid encoding amyloid-beta precursor protein (APP) and/or a nucleic acid encoding presenilin-1 (PSEN1), cells comprising the compositions, animals comprising the compositions, methods of making the cells and animals, methods of modeling Alzheimer's disease (AD), methods of assessing therapeutic candidates for the treatment of Alzheimer's disease or amelioration of symptoms or phenotypes of Alzheimer's disease, and methods of assessing therapeutic candidates for the prevention of Alzheimer's disease or symptoms or phenotypes of Alzheimer's disease.

In one aspect, provided are compositions comprising one or more vectors comprising a nucleic acid encoding amyloid-beta precursor protein and a nucleic acid encoding presenilin-1. In some such compositions, the amyloid-beta precursor protein is an APP695 amyloid-beta precursor protein isoform. In some such compositions, the amyloid-beta precursor protein comprises one or more mutations associated with familial Alzheimer's disease. In some such compositions, the amyloid-beta precursor protein comprises three mutations associated with familial Alzheimer's disease. In some such compositions, the amyloid-beta precursor protein comprises the following mutations with reference to a human APP770 isoform: K670N/M671L; E693G; and I716F. In some such compositions, the amyloid-beta precursor protein comprises K670N/M671L, E693G, and I716F mutations with reference to a human APP770 isoform or K595N/M596L, E618G, and 1641F mutations with reference to a human APP695 isoform. In some such compositions, the amyloid-beta precursor protein is at least 90%, at least 95%, at least 96%, at least 97%, at least 98%, or at least 99% identical to the sequence set forth in SEQ ID NO: 1. In some such compositions, the amyloid-beta precursor protein comprises, consists essentially of, or consist of the sequence set forth in SEQ ID NO: 1. In some such compositions, the nucleic acid encoding the amyloid-beta precursor protein is at least 90%, at least 95%, at least 96%, at least 97%, at least 98%, or at least 99% identical to the sequence set forth in SEQ ID NO: 2. In some such compositions, the nucleic acid encoding the amyloid-beta precursor protein comprises, consists essentially of, or consists of the sequence set forth in SEQ ID NO: 2.

In some such compositions, the presenilin-1 comprises one or more mutations associated with familial Alzheimer's disease. In some such compositions, the presenilin-1 comprises two mutations associated with familial Alzheimer's disease. In some such compositions, the presenilin-1 comprises the following mutations: M146L and L286V. In some such compositions, the presenilin-1 is at least 90%, at least 95%, at least 96%, at least 97%, at least 98%, or at least 99% identical to the sequence set forth in SEQ ID NO: 3. In some such compositions, the presenilin-1 comprises, consists essentially of, or consist of the sequence set forth in SEQ ID NO: 3. In some such compositions, the nucleic acid encoding the presenilin-1 is at least 90%, at least 95%, at least 96%, at least 97%, at least 98%, or at least 99% identical to the sequence set forth in SEQ ID NO: 4. In some such compositions, the nucleic acid encoding the presenilin-1 comprises, consists essentially of, or consists of the sequence set forth in SEQ ID NO: 4.

In some such compositions, the amyloid-beta precursor protein comprises the following mutations with reference to a human APP770 isoform: K670N/M671L; E693G; and 1716F, and the presenilin-1 comprises the following mutations: M146L and L286V. In some such compositions, the amyloid-beta precursor protein comprises K670N/M671L, E693G, and 1716F mutations with reference to a human APP770 isoform or K595N/M596L, E618G, and 1641F mutations with reference to a human APP695 isoform, and the presenilin-1 comprises the following mutations: M146L and L286V. In some such compositions, the amyloid-beta precursor protein is at least 90%, at least 95%, at least 96%, at least 97%, at least 98%, or at least 99% identical to the sequence set forth in SEQ ID NO: 1, and the presenilin-1 is at least 90%, at least 95%, at least 96%, at least 97%, at least 98%, or at least 99% identical to the sequence set forth in SEQ ID NO: 3. In some such compositions, the amyloid-beta precursor protein comprises, consists essentially of, or consist of the sequence set forth in SEQ ID NO: 1, and the presenilin-1 comprises, consists essentially of, or consist of the sequence set forth in SEQ ID NO: 3. In some such compositions, the nucleic acid encoding the amyloid-beta precursor protein is at least 90%, at least 95%, at least 96%, at least 97%, at least 98%, or at least 99% identical to the sequence set forth in SEQ ID NO: 2, and the nucleic acid encoding the presenilin-1 is at least 90%, at least 95%, at least 96%, at least 97%, at least 98%, or at least 99% identical to the sequence set forth in SEQ ID NO: 4. In some such compositions, the nucleic acid encoding the amyloid-beta precursor protein comprises, consists essentially of, or consists of the sequence set forth in SEQ ID NO: 2, and the nucleic acid encoding the presenilin-1 comprises, consists essentially of, or consists of the sequence set forth in SEQ ID NO: 4.

In some such compositions, the nucleic acid encoding the amyloid beta precursor protein is operably linked to a promoter. In some such compositions, the promoter operably linked to the nucleic acid encoding the amyloid beta precursor protein is a constitutive promoter, a tissue-specific promoter, or an inducible promoter. In some such compositions, the promoter operably linked to the nucleic acid encoding the amyloid beta precursor protein is a neuron-specific promoter, optionally wherein the promoter is a synapsin-1 promoter, and optionally wherein the promoter is a human synapsin-1 promoter.

In some such compositions, the nucleic acid encoding the presenilin-1 is operably linked to a promoter. In some such compositions, the promoter operably linked to the nucleic acid encoding the presenilin-1 is a constitutive promoter, a tissue-specific promoter, or an inducible promoter. In some such compositions, the promoter operably linked to the nucleic acid encoding the presenilin-1 is a neuron-specific promoter, optionally wherein the promoter is a synapsin-1 promoter, and optionally wherein the promoter is a human synapsin-1 promoter.

In some such compositions, the nucleic acid encoding the amyloid beta precursor protein is operably linked to a promoter, and the nucleic acid encoding the presenilin-1 is operably linked to a promoter. In some such compositions, the promoter operably linked to the nucleic acid encoding the amyloid beta precursor protein is a constitutive promoter, a tissue-specific promoter, or an inducible promoter, and the promoter operably linked to the nucleic acid encoding the presenilin-1 is a constitutive promoter, a tissue-specific promoter, or an inducible promoter. In some such compositions, the promoter operably linked to the nucleic acid encoding the amyloid beta precursor protein is a neuron-specific promoter, optionally wherein the promoter is a synapsin-1 promoter, and optionally wherein the promoter is a human synapsin-1 promoter, and the promoter operably linked to the nucleic acid encoding the presenilin-1 is a neuron-specific promoter, optionally wherein the promoter is a synapsin-1 promoter, and optionally wherein the promoter is a human synapsin-1 promoter.

In some such compositions, the one or more vectors comprise one or more viral vectors. In some such compositions, the one or more vectors comprise one or more adeno-associated virus (AAV) vectors. In some such compositions, the one or more AAV vectors comprise one or more recombinant AAV9 vectors.

In some such compositions, the amyloid-beta precursor protein comprises the following mutations with reference to a human APP770 isoform: K670N/M671L; E693G; and I716F, optionally wherein the amyloid-beta precursor protein comprises, consists essentially of, or consist of the sequence set forth in SEQ ID NO: 1, the presenilin-1 comprises the following mutations: M146L and L286V, optionally wherein the presenilin-1 comprises, consists essentially of, or consist of the sequence set forth in SEQ ID NO: 3, the nucleic acid encoding the amyloid beta precursor protein is operably linked to a neuron-specific promoter, optionally wherein the promoter is a human synapsin-1 promoter, and the nucleic acid encoding the presenilin-1 is operably linked to a neuron-specific promoter, optionally wherein the promoter is a human synapsin-1 promoter, and the one or more vectors comprise one or more adeno-associated virus (AAV) vectors, optionally wherein the one or more AAV vectors comprise one or more recombinant AAV9 vectors. In some such compositions, the amyloid-beta precursor protein comprises K670N/M671L, E693G, and I716F mutations with reference to a human APP770 isoform or K595N/M596L, E618G, and 1641F mutations with reference to a human APP695 isoform, optionally wherein the amyloid-beta precursor protein comprises, consists essentially of, or consist of the sequence set forth in SEQ ID NO: 1, the presenilin-1 comprises the following mutations: M146L and L286V, optionally wherein the presenilin-1 comprises, consists essentially of, or consist of the sequence set forth in SEQ ID NO: 3, the nucleic acid encoding the amyloid beta precursor protein is operably linked to a neuron-specific promoter, optionally wherein the promoter is a human synapsin-1 promoter, the nucleic acid encoding the presenilin-1 is operably linked to a neuron-specific promoter, optionally wherein the promoter is a human synapsin-promoter, and the one or more vectors comprise one or more adeno-associated virus (AAV) vectors, optionally wherein the one or more AAV vectors comprise one or more recombinant AAV9 vectors.

In some such compositions, the one or more vectors comprise a first vector comprising the nucleic acid encoding the amyloid-beta precursor protein and a second vector comprising the nucleic acid encoding the presenilin-1. In some such compositions, the one or more vectors comprise a single vector comprising the nucleic acid encoding the amyloid-beta precursor protein and the nucleic acid encoding the presenilin-1.

In some such compositions, the composition is for use in creating a non-human animal model of Alzheimer's disease.

In another aspect, provided are cells comprising any of the above compositions. In some such cells, the cell is a mammalian cell. In some such cells, the cell is a non-human primate cell. In some such cells, the cell is a rodent cell, optionally wherein the rodent cell is a mouse cell or a rat cell. In some such cells, the cell is the mouse cell. In some such cells, the cell is a neuron. In some such cells, the cell is in vivo in a subject. In some such cells, the cell is a neuron in the brain of the subject.

In some such cells, the cell comprises a human microtubule-associated protein tau coding sequence comprising a tauopathy-associated mutation, wherein the microtubule-associated protein tau is expressed, optionally wherein the human microtubule-associated protein tau coding sequence is genomically integrated, and optionally wherein the tauopathy-associated mutation is a P301S mutation. In some such cells, (I) the cell comprises a humanized APOE genomic locus, optionally wherein a human apolipoprotein E4 protein is expressed from the humanized APOE genomic locus; (II) the cell comprises a humanized MAPT genomic locus, optionally wherein a human microtubule-associated protein tau protein is expressed from the humanized MAPT genomic locus; (III) the cell comprises a humanized APP genomic locus, optionally wherein a chimeric non-human animal/human amyloid-beta precursor protein comprising K670N/M671L mutations with reference to a human APP770 isoform or K595N/M596L mutations with reference to a human APP695 isoform is expressed from the humanized APP genomic locus; or (IV) any combination thereof. In some such cells, the cell comprises the humanized APOE genomic locus, the humanized MAPT genomic locus, and the humanized APP genomic locus.

In another aspect, provided are non-human animals comprising any of the above compositions. In some such non-human animals, the non-human animal is a mammal. In some such non-human animals, the non-human animal is a non-human primate. In some such non-human animals, the non-human animal is a rodent, optionally wherein the rodent is a mouse or a rat. In some such non-human animals, the non-human animal is the mouse. In some such non-human animals, the non-human animal comprises the composition in the brain. In some such non-human animals, the non-human animal comprises the composition in a plurality of neurons in the brain.

In some such non-human animals, the non-human animal comprises a human microtubule-associated protein tau coding sequence comprising a tauopathy-associated mutation, wherein the microtubule-associated protein tau is expressed, optionally wherein the human microtubule-associated protein tau coding sequence is genomically integrated, and optionally wherein the tauopathy-associated mutation is a P301S mutation. In some such non-human animals, (I) the non-human animal comprises a humanized APOE genomic locus, optionally wherein a human apolipoprotein E4 protein is expressed from the humanized APOE genomic locus; (II) the non-human animal comprises a humanized MAPT genomic locus, optionally wherein a human microtubule-associated protein tau is expressed from the humanized MAPT genomic locus; (III) the non-human animal comprises a humanized APP genomic locus, optionally wherein a chimeric non-human animal/human amyloid-beta precursor protein comprising K670N/M671L mutations with reference to a human APP770 isoform or K595N/M596L mutations with reference to a human APP695 isoform is expressed from the humanized APP genomic locus; or (IV) any combination thereof. In some such non-human animals, the non-human animal comprises the humanized APOE genomic locus, the humanized MAPT genomic locus, and the humanized APP genomic locus.

In some such non-human animals, the non-human animal has one or more Alzheimer's disease phenotypes. In some such non-human animals, the non-human animal has one or more or all of the following: AB plaque pathology; loss of synapses; gliosis; and dystrophic neurites around Aβ plaques. In some such non-human animals, the non-human animal has one or more or all of the following: tau aggregates in dystrophic neurites surrounding AB plaques (NP-tau); neurofibrillary tangles (NFTs); and increased phosphorylated tau in the somatodendritic compartment of neurons. In some such non-human animals, the non-human animal has co-deposition of ApoE protein in Aβ plaques.

In another aspect, provided are methods of modeling Alzheimer's disease in a non-human animal. Some such methods comprise administering any of the above compositions to the non-human animal. In some such methods, the non-human animal is a mammal. In some such methods, the non-human animal is a non-human primate. In some such methods, the non-human animal is a rodent, optionally wherein the rodent is a mouse or a rat. In some such methods, the non-human animal is the mouse.

In some such methods, the composition is administered to the brain of the non-human animal. In some such methods, the composition is administered to the non-human animal via intrahippocampal injection, intracerebroventricular injection, intracranial injection, intrathecal injection, or stereotactic injection. In some such methods, the one or more vectors comprise a first vector comprising the nucleic acid encoding the amyloid-beta precursor protein and a second vector comprising the nucleic acid encoding the presenilin-1, and wherein the first and second vectors are administered simultaneously. In some such methods, the one or more vectors comprise a first vector comprising the nucleic acid encoding the amyloid-beta precursor protein and a second vector comprising the nucleic acid encoding the presenilin-1, and wherein the first and second vectors are administered separately or sequentially.

In some such methods, the non-human animal comprises a human microtubule-associated protein tau coding sequence comprising a tauopathy-associated mutation, wherein the microtubule-associated protein tau is expressed, optionally wherein the human microtubule-associated protein tau coding sequence is genomically integrated, and optionally wherein the tauopathy-associated mutation is a P301S mutation. In some such methods, (I) the non-human animal comprises a humanized APOE genomic locus, optionally wherein a human apolipoprotein E4 protein is expressed from the humanized APOE genomic locus; (II) the non-human animal comprises a humanized MAPT genomic locus, optionally wherein a human microtubule-associated protein tau protein is expressed from the humanized MAPT genomic locus; (III) the non-human animal comprises a humanized APP genomic locus, optionally wherein a chimeric non-human animal/human amyloid-beta precursor protein comprising K670N/M671L mutations with reference to a human APP770 isoform or K595N/M596L mutations with reference to a human APP695 isoform is expressed from the humanized APP genomic locus; or (IV) any combination thereof. In some such methods, the non-human animal comprises the humanized APOE genomic locus, the humanized MAPT genomic locus, and the humanized APP genomic locus.

In some such methods, the non-human animal develops one or more Alzheimer's disease phenotypes after administration of the composition. In some such methods, the non-human animal develops one or more or all of the following after administration of the composition, optionally compared to a control non-human animal that has not been administered the composition: Aβ plaque pathology; loss of synapses; gliosis; and dystrophic neurites around Aβ plaques. In some such methods, the non-human animal develops one or more or all of the following after administration of the composition, optionally compared to a control non-human animal that has not been administered the composition: tau aggregates in dystrophic neurites surrounding Aβ plaques (NP-tau); neurofibrillary tangles (NFTs); and increased phosphorylated tau in the somatodendritic compartment of neurons. In some such methods, the non-human animal develops the following after administration of the composition, optionally compared to a control non-human animal that has not been administered the composition: co-deposition of ApoE protein in Aβ plaques. In some such methods, the non-human animal develops one or more Alzheimer's disease phenotypes within two months after administration of the composition. In some such methods, the non-human animal develops one or more Alzheimer's disease phenotypes within one month after administration of the composition. In some such methods, the non-human animal develops Aβ plaque pathology within one month after administration of the composition.

In another aspect, provided are methods of making any of the above non-human animals. Some such methods comprise administering any of the above compositions to the non-human animal. In some such methods, the non-human animal is a mammal. In some such methods, the non-human animal is a non-human primate. In some such methods, the non-human animal is a rodent, optionally wherein the rodent is a mouse or a rat. In some such methods, the non-human animal is the mouse.

In some such methods, the composition is administered to the brain of the non-human animal. In some such methods, the composition is administered to the non-human animal via intrahippocampal injection, intracerebroventricular injection, intracranial injection, intrathecal injection, or stereotactic injection. In some such methods, the one or more vectors comprise a first vector comprising the nucleic acid encoding the amyloid-beta precursor protein and a second vector comprising the nucleic acid encoding the presenilin-1, and wherein the first and second vectors are administered simultaneously. In some such methods, the one or more vectors comprise a first vector comprising the nucleic acid encoding the amyloid-beta precursor protein and a second vector comprising the nucleic acid encoding the presenilin-1, and wherein the first and second vectors are administered separately or sequentially.

In some such methods, the non-human animal develops one or more Alzheimer's disease phenotypes after administration of the composition. In some such methods, the non-human animal develops one or more or all of the following after administration of the composition, optionally compared to a control non-human animal that has not been administered the composition: Aβ plaque pathology; loss of synapses; gliosis; and dystrophic neurites around Aβ plaques. In some such methods, the non-human animal develops one or more Alzheimer's disease phenotypes within two months after administration of the composition. In some such methods, the non-human animal develops one or more Alzheimer's disease phenotypes within one month after administration of the composition. In some such methods, the non-human animal develops Aβ plaque pathology within one month after administration of the composition.

In another aspect, provided are methods of assessing a therapeutic candidate for the treatment of Alzheimer's disease. Some such methods comprise: (a) administering a candidate agent to any of the above non-human animals; (b) performing one or more assays to determine if the candidate agent has an effect on an Alzheimer's disease phenotype; and (c) identifying the candidate agent that ameliorates the Alzheimer's disease phenotype as a therapeutic candidate. In some such methods, the assessing in step (b) is compared to the non-human animal before administration of the candidate agent or is compared to a control non-human animal that has not been administered the candidate agent. In some such methods, the Alzheimer's disease phenotype comprises one or more of the following: Aβ plaque pathology; loss of synapses; gliosis; and dystrophic neurites around Aβ plaques. In some such methods, the non-human animal is a mammal. In some such methods, the non-human animal is a non-human primate. In some such methods, the non-human animal is a rodent, optionally wherein the rodent is a mouse or a rat. In some such methods, the non-human animal is the mouse.

In another aspect, provided are methods of assessing a therapeutic candidate for the prevention of Alzheimer's disease. Some such methods comprise: (a) administering a candidate agent to a non-human animal; (b) administering any of the above compositions to the non-human animal; (c) performing one or more assays to determine if the candidate agent has an effect on an Alzheimer's disease phenotype; and (d) identifying the candidate agent that prevents the Alzheimer's disease phenotype as a therapeutic candidate. In some such methods, the assessing in step (c) is compared to a control non-human animal that has been administered the composition but has not been administered the candidate agent. In some such methods, the composition is administered to the brain of the non-human animal in step (b). In some such methods, the composition is administered to the non-human animal via intrahippocampal injection, intracerebroventricular injection, intracranial injection, intrathecal injection, or stereotactic injection. In some such methods, step (c) is at least one month after step (b). In some such methods, step (c) is at least two months after step (b). In some such methods, the non-human animal comprises a human microtubule-associated protein tau coding sequence comprising a tauopathy-associated mutation, wherein the microtubule-associated protein tau is expressed, optionally wherein the human microtubule-associated protein tau coding sequence is genomically integrated, and optionally wherein the tauopathy-associated mutation is a P301S mutation. In some such methods, (I) the non-human animal comprises a humanized APOE genomic locus, optionally wherein a human apolipoprotein E4 protein is expressed from the humanized APOE genomic locus; (II) the non-human animal comprises a humanized MAPT genomic locus, optionally wherein a human microtubule-associated protein tau protein is expressed from the humanized MAPT genomic locus; (III) the non-human animal comprises a humanized APP genomic locus, optionally wherein a chimeric non-human animal/human amyloid-beta precursor protein comprising K670N/M671L mutations with reference to a human APP770 isoform or K595N/M596L mutations with reference to a human APP695 isoform is expressed from the humanized APP genomic locus; or (IV) any combination thereof. In some such methods, the non-human animal comprises the humanized APOE genomic locus, the humanized MAPT genomic locus, and the humanized APP genomic locus. In some such methods, the candidate agent is an RNAi agent, an antisense oligonucleotide, an antigen-binding protein, a small molecule, or a nuclease agent. In some such methods, the Alzheimer's disease phenotype comprises one or more of the following: Aβ plaque pathology; loss of synapses; gliosis; and dystrophic neurites around Aβ plaques. In some such methods, the Alzheimer's disease phenotype comprises one or more of the following: tau aggregates in dystrophic neurites surrounding Aβ plaques (NP-tau); neurofibrillary tangles (NFTs); and increased phosphorylated tau in the somatodendritic compartment of neurons. In some such methods, the Alzheimer's disease phenotype comprises co-deposition of ApoE protein in Aβ plaques. In some such methods, the non-human animal is a mammal. In some such methods, the non-human animal is a non-human primate. In some such methods, the non-human animal is a rodent, optionally wherein the rodent is a mouse or a rat. In some such methods, the non-human animal is the mouse.

### BRIEF DESCRIPTION OF THE FIGURES

The patent application file contains at least one drawing executed in color. Copies of this patent application with color drawing(s) will be provided by the Office upon request and payment of the necessary fec.

FIG. 1 shows a schematic of a mouse model with a rapid onset of Alzheimer's pathology involving the injection of AAV vectors with human familial Alzheimer's disease (FAD) mutant forms of amyloid-beta precursor protein (APP) and presenilin-1 (PSEN1) directly into the brain of wild type mice.

FIG. 2 shows the part of the sequence of human APP695 comprising Swedish [KM595/596NL], Arctic [E618G], and Iberian [1641F] FAD mutations.

FIG. 3 shows a schematic of the timeline used for the experimental model involving AAV injection of 1-month-old mice and histology performed 1, 2, and 4 months post-injection.

FIGS. 4A-4C show an age-dependent accumulation of amyloid plaques in mice injected with AAV-APP and PSEN1. The Aβ plaques were detected with a β-amyloid antibody (Cell Signaling D54D2). FIG. 4A shows a cross section of brain tissue one month after injection. FIG. 4B shows a cross section of brain tissue two months after injection. FIG. 4C shows a cross section of brain tissue four months after injection.

FIGS. 5A-5C show amyloid plaques present in 3-month-old female C57BL6/J mice two months after AAV injection. The Aβ plaques were detected with a β-amyloid antibody (Cell Signaling D54D2). FIG. 5A shows the injection site in the hippocampus. FIG. 5B shows a cross section of brain tissue with Aβ plaques in the hippocampus. FIG. 5C shows an enlarged view of the hippocampus from FIG. 5B.

FIGS. 6A-6C show dense core and filamentous plaques observed in brain tissue of mice two months after AAV injection. FIG. 6A shows dense core β-sheet deposits detected with methoxy-X04. FIG. 6B shows filamentous plaques detected by staining with an Aβ antibody (Cell Signaling D54D2). FIG. 6C shows a merged image with both methoxy-X04 and Aβ plaque detection.

FIGS. 7A-7C show dystrophic neurites around plaques observed in the brain tissue of mice two months after AAV injection. FIG. 7A shows dense core β-sheet deposits detected with methoxy-X04. FIG. 7B shows staining for Lamp1, a lysosomal protein highly enriched in dystrophic neurites. FIG. 7C shows a merged image with both methoxy-X04 and Lamp1 detection.

FIGS. 8A-8C show N-APP in dystrophic neurites around plaques observed in the brain tissue of mice two months after AAV injection. FIG. 8A shows dense core β-sheet deposits detected with methoxy-X04. FIG. 8B shows staining for N-APP, which is highly enriched in dystrophic neurites. FIG. 8C shows a merged image with both methoxy-X04 and N-APP detection.

FIGS. 9A-9C show microglia clusters around amyloid plaques observed in the hippocampus of mice two months after AAV injection. FIG. 9A shows clusters of microglia (circled) detected by staining for Iba1, a microglial marker. FIG. 9B shows filamentous plaques detected by staining with an Aβ antibody (Cell Signaling D54D2). FIG. 9C shows a merged image with both Iba1 and Aβ plaque detection.

FIGS. 10A-10C show microglia in the cortex of mice two months after AAV injection where no plaques had developed. FIG. 10A show microglia detected by staining for Iba1. FIG. 10B shows cortex tissue stained with an Aβ antibody (Cell Signaling D54D2) with no observable Aβ plaques. FIG. 10C shows a merged image with both Iba1 and Aβ plaque detection.

FIGS. 11A-11B show that microglia exhibit morphological changes in the hippocampus where there are plaques, detected by staining for Iba1. Histology was performed on mice two months after AAV injection. FIG. 11A shows microglia in the hippocampus containing Aβ plaques and FIG. 11B shows microglia in the cortex without Aβ plaques.

FIGS. 12A-12J show that disease-associated microglia signatures were observed in tissue/microglia associated with Aβ plaques. RNA-Seq analysis was performed on tissue/microglial cells from the hippocampus containing Aβ plaques and compared to tissue/microglial cells from the cortex without Aβ plaques from mice four months after AAV injection. Expression of Trem2 (FIG. 12A), Tyrobp (FIG. 12B), Apoc in microglia (FIG. 12C), Itgax (FIG. 12D), Clec7a (FIG. 12E), Csf1 (FIG. 12F), and Cst7 (FIG. 12G) was significantly elevated in the presence of Aβ plaques. Expression of Tmem 119 (FIG. 12H), P2ry12 (FIG. 12I), and Cx3crl (FIG. 12J) was not significantly different based on the presence of Aβ plaques.

FIGS. 13A-13H show that microglia associated with Aβ plaques exhibit signatures of late response microglia. RNA-Seq analysis was performed on tissue/microglial cells from the hippocampus containing Aβ plaques and compared to tissue/microglial cells from the cortex without Aβ plaques from mice four months after AAV injection. Expression of complement components C4b (FIG. 13A) and C3 (FIG. 13B), MHC-I component H2-D1 (FIG. 13C), MHC-II components H2-Aa (FIG. 13D), H2-Ab1 (FIG. 13E), and Cd74 (FIG. 13F), and interferon response genes Ifitm3 (FIG. 13G) and Irf7 (FIG. 13H) were significantly elevated in the presence of Aβ plaques.

FIGS. 14A-14B show elevated detection of glial fibrillary acidic protein (GFAP) in the presence of Aβ plaques, detected with methoxy-X04 in mice two months after AAV injection. FIG. 14A shows the ipsilateral side of the hippocampus with Aβ plaques. FIG. 14B shows the contralateral side of the hippocampus where plaques were not observed.

FIGS. 15A-15D show that disease-associated astrocyte (DAA) signatures were observed in astrocytes associated with Aβ plaques. RNA-Seq analysis was performed on tissue/astrocyte cells from the hippocampus containing Aβ plaques and compared to tissue/astrocyte cells from the cortex without Aβ plaques from mice four months after AAV injection. Expression of Gfap (FIG. 15A), Vim (FIG. 15B), Osmr (FIG. 15C), and Serpina3n (FIG. 15D) was significantly elevated in the presence of Aβ plaques.

FIGS. 16A-16C show evidence of T cell infiltration in mice two months after AAV injection. FIG. 16A shows DAPI staining of a cross section of brain tissue. FIG. 16B shows CD3 staining indicating the presence of T cells. FIG. 16C shows an enlarged image of CD3 staining from FIG. 16B.

FIGS. 17A-17B show T cell infiltration in the presence of Aβ plaques in mice two months after AAV injection. FIG. 17A shows a cross section of the hippocampus stained with an Aβ antibody (Cell Signaling D54D2), revealing the presence of Aβ plaques. FIG. 17B shows CD3 staining indicating the presence of T cells. FIG. 17C shows an expanded view of FIG. 17B highlighting the CD3 staining (circle and arrows).

FIGS. 18A-18C shows a loss of synaptic markers in areas covered by amyloid plaques in the hippocampus of mice two months after injection with AAV. FIG. 18A shows staining for synaptophysin, a presynaptic vesicle protein. FIG. 18B shows Aβ plaques detected with methoxy-X04. FIG. 18C shows a merged image with both synaptophysin and Aβ plaque detection.

FIGS. 19A-19D show that Aβ plaques induced NP-tau and enhanced p-tau in a P301S tauopathy mouse model. FIG. 19A shows a schematic of an experiment in which AAV vectors with human FAD mutant forms of APP and PSEN1 were injected into the hippocampus of the tau P301S mouse model. FIG. 19B shows that mice injected with AAV-APP/PS1 exhibited robust amyloid plaque pathology and the presence of plaque-associated phosphorylated tau pathology, which is located in the peri-plaque region and co-localized with dystrophic neurite marker Lamp1. FIG. 19C shows neuritic plaque-tau pathology (NP-tau) and the classical phosphorylated-tau accumulation in the cytoplasm, known as neurofibrillary tangles (NFT).

FIG. 19D shows that mice injected with AAV-APP/PS1 exhibited higher phosphorylated-tau signals.

FIGS. 20A-20D show that prophylactic Aβ antibody (Aducanumab [mIgG2a]) treatment reduced amyloid plaques and dystrophic neurites. FIG. 20A shows a schematic of an experiment in which AAV vectors with human FAD mutant forms of APP and PSEN1 were injected into the hippocampus of wild type mice. Antibody treatment was started 1 week post-AAV injection to induce amyloid plaque deposition. Antibody was administered weekly for 7 weeks before taking down the mice 1 week after the final antibody administration. FIG. 20B shows that Aducanumab treatment significantly reduced plaque levels compared to mice treated with control antibody. FIG. 20C shows that Aducanumab treatment also significantly reduced plaque-associated dystrophic neurite marker Lamp1 (lysosome aggregation in axons labeled by Lamp1). FIG. 20D shows that Aducanumab treatment did not change the levels of APP and intracellular APP C-terminal fragments.

FIGS. 21A-21D show that rapid accumulation of Aβ plaques can be induced in various mouse models, including mice humanized for APOE, to model AD pathology and test different therapeutic approaches. FIG. 21A shows a schematic for an experiment showing that AAV-mediated expression of mutant APP and PSEN1 facilitated plaque formation. Intrahippocampal injection of AAV, to facilitate neuronal expression of APP and PSEN1 harboring familial AD-associated mutations, resulted in amyloid-β accumulation and plaque formation in mice expressing human APOE. FIG. 21B shows that following AAV injection, APOE4 humanized mice accumulated Aβ plaques and plaque-associated dystrophic neurites, a marker of neuronal damage. FIG. 21C shows that APOE4 humanized mice developed fibrillar Aβ plaques, with β-sheet structure. FIG. 21D shows that in APOE4 humanized mice, ApoE co-deposited with Aβ plaques.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous APP sequence of an animal refers to a native APP sequence that naturally occurs at the APP locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values+5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are compositions comprising a nucleic acid encoding amyloid-beta precursor protein (APP) and/or a nucleic acid encoding presenilin-1 (PSEN1), cells comprising the compositions, animals comprising the compositions, methods of making the cells and animals, methods of modeling Alzheimer's disease (AD), methods of assessing therapeutic candidates for the treatment of AD or amelioration of symptoms or phenotypes of AD, and methods of assessing therapeutic candidates for the prevention of AD or symptoms or phenotypes of AD.

In the Alzheimer's disease field, there are transgenic mouse models that express APP and/or PSEN1/PSEN2 with familial Alzheimer's disease (FAD) mutations. While these mouse models are extremely useful, they have significant disadvantages. First, it is very time-consuming to study how different genes affect AD pathology in the transgenic AD models. It requires breeding of these mouse models to other genetic mouse models, usually taking a year to generate the compound genetic mice. Then, the compound genetic mice must be aged to see the phenotypes. Second, the pathology in the transgenic AD models takes a significant amount of time to appear. Even in the most aggressive model (5×FAD), most of the pathology does not appear until about 6 months of age. Third, existing transgenic AD models are predominantly mice. Models that can be extended to animals closer to humans, such as non-human primates, would be useful.

The models disclosed herein can be generated very quickly, show AD phenotypes within one month, and can be adapted to other non-human animal species. For example, in one non-limiting example, recombinant AAV vectors that express human APP, specifically the APP695 isoform, with 3 FAD mutations (Swedish [KM595/596NL], Arctic [E618G], and Iberian [1641F]), and human PSEN1 with 2 FAD mutations (M146L and L286V), each driven by human synapsin-1 promoters, can be injected into the brain of mice. Upon injection of the AAV vectors into mouse brain, Aβ plaque pathology is observed within one month, and very robust pathology in the injected brain regions is observed after two months. In addition to Aβ plaque pathology, other Alzheimer's-related phenotypes are observed, including loss of synapses, gliosis, dystrophic neurites around plaques, and intracellular lysosome aggregation around plaques, all of which have been reported in postmortem human AD brains.

### II. Non-Human Animal Models of Alzheimer's Disease and Compositions for Use in Making the Non-Human Animal Models

Provided herein are compositions comprising a nucleic acid encoding amyloid-beta precursor protein (APP) and/or a nucleic acid encoding presenilin-1 (PSEN1), cells comprising the compositions, and animals (e.g., non-human animals) comprising the compositions. The compositions are useful for modeling Alzheimer's disease (AD).

AD is a neurodegenerative disorder characterized by progressive dementia, loss of cognitive abilities, and deposition of fibrillar amyloid proteins as intraneuronal neurofibrillary tangles, extracellular amyloid plaques, and vascular amyloid deposits. A hallmark of AD is the aggregation of β-amyloid peptides (Aβ) into amyloid plaques in patient brain. The major constituents of these plaques are neurotoxic amyloid-beta (Aβ) protein 40 and amyloid-beta (Aβ) protein 42, that are produced by the proteolysis of the transmembrane amyloid-beta precursor protein (APP). Cleavage of amyloid precursor protein (APP) by the intramembrane protease γ-secretase produces Aβ of varying lengths, of which longer peptides such as Aβ42 are thought to be more harmful. Increased ratios of longer Aβs over shorter ones, exemplified by the ratio of Aβ42 over Aβ40, may lead to formation of amyloid plaques and consequent development of AD.

Mutations in the PSEN1 gene, encoding presenilin-1 (PS1), are the most common cause of familial Alzheimer's disease (FAD). PS1 functions as the catalytic subunit of γ-secretase, an intramembranous protease that cleaves a variety of type 1 transmembrane proteins, notably including APP and Notch. Following prior cleavage by β-secretase, processing of APP by γ-secretase generates β-amyloid (Aβ) peptides of varying lengths. Whereas Aβ40 accounts for ˜90% of Aβ production, the minor Aβ42 product is more hydrophobic and is thought to nucleate Aβ aggregation, leading to amyloid plaque deposition in the AD brain.

Amyloid-beta precursor proteins and nucleic acids and vectors encoding amyloid-beta precursor proteins are described in more detail below. Likewise, presenilin-1 proteins and nucleic acids and vectors encoding presenilin-1 proteins are described in more detail below, as are cells and non-human animals comprising the proteins or nucleic acids or vectors.

### A. Amyloid-Beta Precursor Protein and Nucleic Acids Encoding

Genetic, biochemical, and behavioral research suggest that physiologic generation of the neurotoxic Aβ peptide from sequential amyloid precursor protein (APP) proteolysis is the crucial step in the development of AD. APP is a single-pass transmembrane protein expressed at high levels in the brain and metabolized in a rapid and highly complex fashion by a series of sequential proteases, including the intramembranous γ-secretase complex, which also process other key regulatory molecules. Alternate splicing of the APP transcript generates 8 isoforms, of which 3 are most common: the 695 (APP695) amino acid form, which is expressed predominantly in the CNS, and the 751 (APP751) and 770 (APP770) amino acid forms, which are more ubiquitously expressed.

The amyloid-beta precursor proteins described herein can be human amyloid-beta precursor proteins, such as human APP770, APP751, or APP695 isoforms. In a specific example, the amyloid-beta precursor protein described herein can be an APP695 isoform. Human amyloid-beta precursor protein (also called APP, ABPP, APPI, Alzheimer disease amyloid A4 protein homolog, Alzheimer disease amyloid protein, amyloid precursor protein, amyloid-beta (A4) precursor protein, amyloid-beta A4 protein, cerebral vascular amyloid peptide (CVAP), PreA4, or protease nexin-II (PN-II)) is assigned UniProt reference number P05067. The human gene encoding amyloid-beta precursor protein (APP, A4, or AD1) is assigned NCBI GeneID 351. The canonical isoform (APP770) is assigned UniProt reference number P05067-1 and NCBI reference number NP_000475.1 (SEQ ID NO: 7). An exemplary coding sequence is assigned reference number CCDS13576.1 (SEQ ID NO: 8). The APP695 isoform expressed predominantly in the CNS is assigned UniProt reference number P05067-4 and NCBI reference number NP_958817.1 (SEQ ID NO: 5). An exemplary coding sequence is assigned reference number CCDS13577.1 (SEQ ID NO: 6). An exemplary amyloid-beta precursor protein (APP695) comprising K670N/M671L, E693G, and I716F mutations (with reference to the canonical APP770 isoform) is set forth in SEQ ID NO: 1. An exemplary coding sequence is set forth in SEQ ID NO: 2. Amyloid-beta precursor proteins from other species are also well-characterized. Mouse amyloid-beta precursor protein is assigned UniProt reference number P12023. Rat amyloid-beta precursor protein is assigned UniProt reference number P08592. Cynomolgus amyloid-beta precursor protein is assigned UniProt reference number P53601.

The amyloid-beta precursor proteins described herein can comprise one or more mutations associated with AD (e.g., FAD). For example, the amyloid-beta precursor proteins described herein can comprise three mutations associated with AD (e.g., FAD). Mutations in amyloid-beta precursor protein are associated with familial forms of early onset AD as well as with Cerebral Amyloid Angiopathy (CAA). Pathogenic mutations generally alter processing by secretases, leading in an overall increase in Aβ production and/or a change in the ratio of specific Aβ peptides. See alzforum.org/mutations/app, TCW et al. (2017) Cold Spring Harb. Perspect Med. 7 (6): a024539, and Dai et al. (2018) Oncotarget 9:15132-15143, each of which is herein incorporated by reference in its entirety for all purposes. In a specific example, an amyloid-beta precursor protein described herein comprises a K670N/M671L mutation (with reference to the canonical human APP770 isoform, or K595N/M596L mutation with reference to the APP695 isoform), which is also known as the Swedish mutation. By K670N/M671L mutation is meant a K670N/M671L mutation in the canonical human APP770 isoform or a corresponding mutation in another amyloid-beta precursor protein when optimally aligned with the canonical human amyloid-beta precursor protein (e.g., a K595N/M596L mutation in the human APP695 isoform). In a specific example, an amyloid-beta precursor protein described herein comprises an E693G mutation (with reference to the canonical human APP770 isoform, or E618G mutation with reference to the APP695 isoform), which is also known as the Arctic mutation. By E693G mutation is meant an E693G mutation in the canonical human APP770 isoform or a corresponding mutation in another amyloid-beta precursor protein when optimally aligned with the canonical human amyloid-beta precursor protein (e.g., an E618G mutation in the human APP695 isoform). In a specific example, an amyloid-beta precursor protein described herein comprises an 1716F mutation (with reference to the canonical human APP770 isoform, or 1641F mutation with reference to the APP695 isoform), which is also known as the Iberian mutation. By I716F mutation is meant an I716F mutation in the canonical human APP770 isoform or a corresponding mutation in another amyloid-beta precursor protein when optimally aligned with the canonical human amyloid-beta precursor protein (e.g., an 1641F mutation in the human APP695 isoform). In another specific example, an amyloid-beta precursor protein described herein (e.g., APP695) comprises a K670N/M671L mutation, an E693G mutation, and an I716F mutation. Such an amyloid-beta precursor protein can comprise a sequence at least 90%, at least 95%, at least 96%, at least 97%, at least 98%, at least 99%, or 100% identical to SEQ ID NO: 1. For example, the amyloid-beta precursor protein can comprise, consist essentially of, or consist of the sequence set forth in SEQ ID NO: 1. A nucleic acid encoding such an amyloid-beta precursor protein can comprise a sequence at least 90%, at least 95%, at least 96%, at least 97%, at least 98%, at least 99%, or 100% identical to SEQ ID NO: 2. For example, the nucleic acid can comprise, consist essentially of, or consist of the sequence set forth in SEQ ID NO: 2.

The Swedish mutation (K670N/M671L) repeatedly has been shown to increase total Aβ levels by facilitating amyloid-beta precursor protein cleavage by BACE1. In carriers of the Swedish mutation, both Aβ40 and Aβ42 are elevated in the plasma. See, e.g., Shin et al. (2010) BMB Rep. 43 (10:704-709, herein incorporated by reference in its entirety for all purposes. Aβ40 produced from amyloid-beta precursor protein with the Arctic mutation (E693G) has an increased propensity to form protofibrils and does so at a faster rate compared with wild-type Aβ40. See, e.g., Nilsberth et al. (2001) Nat. Neurosci. 4 (9): 887-893 and Kamino et al. (1992) Am. J. Hu. Genet. 51 (5): 998-1014, each of which is herein incorporated by reference in its entirety for all purposes. The Iberian mutation (1716F) is associated with extensive and often mixed neuropathology, characterized by typical AD pathology (e.g., amyloid plaques and neurofibrillary tangles), in addition to α-synuclein pathology in some cases. This mutation affects APP cleavage by γ-secretase. Specifically, the mutation affects γ-secretase cleavage specificity and causes a dramatic increase in the Aβ42/Aβ40 ratio. See, e.g., Eckman et al. (1997) Hum. Mol. Genet. 6 (12): 2087-2089, herein incorporated by reference in its entirety for all purposes.

Also provided herein are nucleic acids or nucleic acid constructs encoding amyloid-beta precursor proteins. The nucleic acids or nucleic acid constructs can be isolated nucleic acid constructs. The nucleic acids or nucleic acid constructs can be in a vector as described in more detail elsewhere herein.

In some cases, the nucleic acid encoding the amyloid-beta precursor protein can be codon-optimized (e.g., codon-optimized for expression in a human or expression in a mouse). For example, the nucleic acid can be modified to substitute codons having a higher frequency of usage in a human cell, a mammalian cell, a non-human primate cell, a rodent cell, a mouse cell, a rat cell, or any other host cell of interest.

The nucleic acid encoding the amyloid-beta precursor protein can be DNA or RNA. The nucleic acid in some cases can be a messenger RNA (mRNA) encoding the amyloid-beta precursor protein. The nucleic acid in some cases can be a complementary DNA (cDNA) encoding the amyloid-beta precursor protein. Examples of coding sequences for some of the amyloid-beta precursor proteins described herein are set forth, e.g., in SEQ ID NOS: 2, 6, and 8. For example, such nucleic acids may contain only coding sequence without any intervening introns. In other cases, the nucleic acid can comprise one or more introns separating exons in the amyloid-beta precursor protein coding sequence. For example, the nucleic acid can comprise genomic sequence including both exons and introns.

In some cases, the nucleic acid is in an expression construct comprising the nucleic acid encoding the amyloid-beta precursor protein operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In some cases, the promoter is a heterologous promoter (i.e., a promoter to which an amyloid-beta precursor protein nucleic acid is not operably linked naturally). In other cases, the promoter can be an endogenous promoter (i.e., amyloid-beta precursor protein nucleic acid operably linked to an amyloid-beta precursor protein promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter).

The nucleic acids and expression constructs disclosed herein can also comprise post-transcriptional regulatory elements, such as the woodchuck hepatitis virus post-transcriptional regulatory element. The nucleic acids and expression constructs can further comprise one or more polyadenylation signal sequences. For example, the nucleic acid construct can comprise a polyadenylation signal sequence located 3′ of the amyloid-beta precursor protein coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOXI transcription termination sequence, a CYC1 transcription termination sequence, or any transcription termination sequence known to be suitable for regulating gene expression in eukaryotic cells.

The nucleic acids and expression constructs can also comprise a polyadenylation signal sequence upstream of the amyloid-beta precursor protein coding sequence. The polyadenylation signal sequence upstream of the amyloid-beta precursor protein coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the amyloid-beta precursor protein encoded by the nucleic acid or expression constructs can be expressed in a neuron-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). For example, modifications can be made to one or more nucleosides within an mRNA. Examples of chemical modifications to mRNA nucleobases include pseudouridine, 1-methyl-pseudouridine, and 5-methyl-cytidine. mRNA can also be capped. mRNA can also be polyadenylated (to comprise a poly(A) tail). As one example, capped and polyadenylated mRNA containing N1-methyl-pseudouridine can be used (e.g., can be fully substituted with N1-methyl-pseudouridine). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (cGFP), cyan fluorescent protein (CFP), yellow fluorescent protein (YFP), enhanced yellow fluorescent protein (eYFP), blue fluorescent protein (BFP), enhanced blue fluorescent protein (cBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

### B. Presenilin-1 and Nucleic Acids Encoding

PSEN1 encodes presenilin-1, a subunit of γ-secretase, the aspartyl protease responsible for Aβ generation. Presenilin-1 functions as the catalytic subunit of γ-secretase, an intramembranous protease that cleaves a variety of type 1 transmembrane proteins, notably including the amyloid precursor protein (APP) and Notch. Following prior cleavage by β-secretase, processing of APP by γ-secretase generates β-amyloid (Aβ) peptides of varying lengths. Whereas Aβ40 accounts for ˜90% of Aβ production, the minor Aβ42 product is more hydrophobic and is thought to nucleate Aβ aggregation, leading to amyloid plaque deposition in the AD brain.

The presenilin-1 proteins described herein can be human presenilin-1 proteins. Human presenilin-1 (also called PS1, PS-1, or Protein S182) is assigned UniProt reference number P49768. The human gene encoding presenilin-1 (PSEN1, AD3, PS1, or PSNL1) is assigned NCBI GeneID 5663. The canonical isoform is assigned UniProt reference number P-49768-1 and NCBI reference number NP_000012.1 (SEQ ID NO: 9). An exemplary coding sequence is assigned reference number CCDS9812.1 (SEQ ID NO: 10). An exemplary presenilin-1 protein comprising M146L and L286V mutations is set forth in SEQ ID NO: 3. An exemplary coding sequence is set forth in SEQ ID NO: 4. Presenilin-1 proteins from other species are also well-characterized. Mouse presenilin-1 is assigned UniProt reference number P49769. Rat presenilin-1 is assigned UniProt reference number P97887. Cynomolgus presenilin-1 is assigned UniProt reference number Q8HXW5.

The presenilin-1 proteins described herein can comprise one or more mutations associated with AD (e.g., FAD). For example, the presenilin-1 proteins described herein can comprise two mutations associated with AD (e.g., FAD). Mutations in the PSEN1 gene are the most common cause of FAD. More than 300 mutations in PSEN1 have been reported. See alzforum.org/mutations/psen-1, Kelleher et al. (2017) Proc. Natl. Acad. Sci. U.S.A. 114 (4): 629-631, and Dai et al. (2018) Oncotarget 9:15132-15143, each of which is herein incorporated by reference in its entirety for all purposes. In a specific example, a presenilin-1 protein described herein comprises a M146L mutation. By M146L mutation is meant a M146L mutation in the canonical human presenilin-1 protein or a corresponding mutation in another presenilin-1 protein when optimally aligned with the canonical human presenilin-1 protein. In another specific example, a presenilin-1 protein described herein comprises a L286V mutation. By L286V mutation is meant a L286V mutation in the canonical human presenilin-1 protein or a corresponding mutation in another presenilin-1 protein when optimally aligned with the canonical human presenilin-1 protein. In another specific example, a presenilin-1 protein described herein comprises a M146L mutation and a L286V mutation. Such a presenilin-1 protein can comprise a sequence at least 90%, at least 95%, at least 96%, at least 97%, at least 98%, at least 99%, or 100% identical to SEQ ID NO: 3. For example, the presenilin-1 protein can comprise, consist essentially of, or consist of the sequence set forth in SEQ ID NO: 3. A nucleic acid encoding such a presenilin-1 protein can comprise a sequence at least 90%, at least 95%, at least 96%, at least 97%, at least 98%, at least 99%, or 100% identical to SEQ ID NO: 4. For example, the nucleic acid can comprise, consist essentially of, or consist of the sequence set forth in SEQ ID NO: 4.

The M146L mutation alters the conformation of the active site (slightly increased protease activity with APP; decreased activity for Notch1 cleavage; no loss of its ability to cleave Ephb2/CTF1). See, e.g., Sun et al. (2017) Proc. Natl. Acad. Sci. U.S.A. 114 (4): E476-E485, Campion et al. (1999) Am. J. Hum. Genet. 65 (3): 664-670, Bruni et al. (2010) Neurology 74 (10): 798-806, Chau et al. (2012) J. Biol. Chem. 287 (21): 17288-17296, herein incorporated by reference in its entirety for all purposes. The L286V mutation results in altered amyloid-beta production and increased amyloid-beta 42/amyloid-beta 40 ratio. See, e.g., Sun et al. (2017) Proc. Natl. Acad. Sci. U.S.A. 114 (4): E476-E485, herein incorporated by reference in its entirety for all purposes.

Also provided herein are nucleic acids or nucleic acid constructs encoding presenilin-1 proteins. The nucleic acids or nucleic acid constructs can be isolated nucleic acid constructs. The nucleic acids or nucleic acid constructs can be in a vector as described in more detail elsewhere herein.

In some cases, the nucleic acid encoding the presenilin-1 protein can be codon-optimized (e.g., codon-optimized for expression in a human or expression in a mouse). For example, the nucleic acid can be modified to substitute codons having a higher frequency of usage in a human cell, a mammalian cell, a non-human primate cell, a rodent cell, a mouse cell, a rat cell, or any other host cell of interest.

The nucleic acid encoding the presenilin-1 protein can be DNA or RNA. The nucleic acid in some cases can be a messenger RNA (mRNA) encoding the presenilin-1 protein. The nucleic acid in some cases can be a complementary DNA (cDNA) encoding the presenilin-1 protein. Examples of coding sequences for some of the presenilin-1 proteins described herein are set forth, e.g., in SEQ ID NOS: 4 and 10. For example, such nucleic acids may contain only coding sequence without any intervening introns. In other cases, the nucleic acid can comprise one or more introns separating exons in the presenilin-1 protein coding sequence. For example, the nucleic acid can comprise genomic sequence including both exons and introns.

In some cases, the nucleic acid is in an expression construct comprising the nucleic acid encoding the presenilin-1 protein operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In some cases, the promoter is a heterologous promoter (i.e., a promoter to which a presenilin-1 nucleic acid is not operably linked naturally). In other cases, the promoter can be an endogenous promoter (i.e., presenilin-1 nucleic acid operably linked to a presenilin-1 promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter).

The nucleic acids and expression constructs disclosed herein can also comprise post-transcriptional regulatory elements, such as the woodchuck hepatitis virus post-transcriptional regulatory element. The nucleic acids and expression constructs can further comprise one or more polyadenylation signal sequences. For example, the nucleic acid construct can comprise a polyadenylation signal sequence located 3′ of the presenilin-1 protein coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOXI transcription termination sequence, a CYC1 transcription termination sequence, or any transcription termination sequence known to be suitable for regulating gene expression in eukaryotic cells.

The nucleic acids and expression constructs can also comprise a polyadenylation signal sequence upstream of the presenilin-1 protein coding sequence. The polyadenylation signal sequence upstream of the presenilin-1 protein coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the presenilin-1 protein encoded by the nucleic acid or expression constructs can be expressed in a neuron-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). For example, modifications can be made to one or more nucleosides within an mRNA. Examples of chemical modifications to mRNA nucleobases include pseudouridine, 1-methyl-pseudouridine, and 5-methyl-cytidine. mRNA can also be capped. mRNA can also be polyadenylated (to comprise a poly(A) tail). As one example, capped and polyadenylated mRNA containing N1-methyl-pseudouridine can be used (e.g., can be fully substituted with N1-methyl-pseudouridine). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (cBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

### C. Vectors

Also provided herein are vectors comprising the nucleic acids, nucleic acid constructs, or expression constructs encoding amyloid-beta precursor protein and/or presenilin-1. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The nucleic acids or expression constructs can be in a vector, such as a viral vector. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include about 10¹², about 10¹³, about 10¹⁴, about 10¹⁵, and about 10¹⁶ vector genomes/mL (e.g., about 10¹³ vector genomes/mL). Other exemplary viral titers (e.g., AAV titers) include about 10¹², about 10¹³, about 10¹⁴, about 10¹⁵, and about 10¹⁶ vector genomes (vg)/kg of body weight (e.g., about 10¹³ vg/kg).

In one example, the nucleic acid or expression construct is in an AAV vector (i.e., a recombinant AAV vector). The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2α, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, AAV9, and AAV10. Selectivity of AAV serotypes for gene delivery in neurons is discussed, for example, in Hammond et al. (2017) PLOS One 12 (12): e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### D. Lipid Nanoparticles

Also provided herein are lipid nanoparticles comprising the amyloid-beta precursor proteins and/or presenilin-1 proteins or the nucleic acids, nucleic acid constructs, expression constructs, or vectors encoding the amyloid-beta precursor proteins and/or presenilin-1 proteins.

Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22 (9): 2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

### E. Cells or Animals

Cells or subjects (e.g., animals or non-human animals) comprising the amyloid-beta precursor protein and/or presenilin-1 or the nucleic acids, nucleic acid constructs, expression constructs, vectors, or lipid nanoparticles disclosed herein are also provided. The cells or subjects can express the amyloid-beta precursor protein and/or presenilin-1.

The cells or subjects can be, for example, non-human animal, mammalian, non-human mammalian, and human. A mammal can be, for example, a non-human mammal, a human, a non-human primate, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans. In one specific example, the non-human animal can be a non-human primate. In another specific example, the non-human animal can be a rodent. In another specific example, the non-human animal can be a mouse. In another specific example, the non-human animal can be a rat.

Cells can be isolated cells (e.g., in vitro) or can be in vivo within a subject (e.g., animal or mammal). Cells can also be any type of undifferentiated or differentiated state. In one example, the cells are neurons. The cells provided herein can be normal, healthy cells, or can be diseased cells (e.g., Aβ plaque pathology).

In one example, the cell is a human cell, a non-human primate cell, a rodent cell, a mouse cell, or a rat cell such as a human neuron, a non-human primate neuron, a rodent neuron, a mouse neuron, or a rat neuron. In a specific example, the cell is a non-human primate neuron. In another specific example, the cell is a mouse neuron. In another specific example, the cell is a rat neuron. In a specific example, the cell is in vivo in a subject (e.g., a neuron in the brain of a subject).

The cells or animals (e.g., non-human animals) disclosed herein can have one or more symptoms or phenotypes of AD. For example, an animal disclosed herein can have one or more or all of the following: Aβ plaque pathology; loss of synapses; gliosis; dystrophic neurites around Aβ plaques; and intracellular lysosome aggregation around Aβ plaques. As another example, an animal disclosed herein can have one or more or all of the following: tau aggregates in dystrophic neurites surrounding Aβ plaques (NP-tau); neurofibrillary tangles (NFTs); and increased phosphorylated tau in the somatodendritic compartment of neurons. As another example, an animal disclosed herein can have the following: co-deposition of ApoE (e.g., human ApoE, or human ApoE4) protein in Aβ plaques. These phenotypes can be increased, for example, compared to a control cell or animal that does not comprise the amyloid-beta precursor protein and/or presenilin-1 or the nucleic acids, nucleic acid constructs, expression constructs, vectors, or lipid nanoparticles disclosed herein.

In some embodiments, the cells or animals can be wild type cells or animals. In other embodiments, the cells or animals can express one or more mutant proteins (e.g., can comprise one or more mutant genes) and/or can express one or more human proteins or chimeric non-human animal/human proteins (e.g., can comprise one or more humanized genes).

In one example, the cells or animals can express a microtubule-associated protein tau (tau) protein with a tauopathy-associated mutation, such as a tau P301S mutation. For example, the cells or animals can express the tau protein in neurons. Microtubule-associated protein tau (also called neurofibrillary tangle protein, paired helical filament-tau (PHF-tau), or tau) is a protein that promotes microtubule assembly and stability and is predominantly expressed in neurons, where it is preferentially localized to the axonal compartment. Tau is encoded by the MAPT gene (also called MAPTL, MTBT1, TAU, or MTAPT). Tau has a role in stabilizing neuronal microtubules and thus in promoting axonal outgrowth. In humans, it appears as a set of six isoforms which are differentially spliced from transcripts of a single gene located on chromosome 17. Each tau isoform contains a series of 3/4 tandem repeat units (depending on the isoform) that bind to microtubules and serve to stabilize them. The microtubule-binding repeat region of tau is flanked by serine/threonine-rich regions which can be phosphorylated by a variety of kinases and that are associated with tau hyperphosphorylation in AD and a family of related neurodegenerative diseases called tauopathies. In AD and other tauopathies, tau protein is abnormally hyperphosphorylated and aggregated into bundles of filaments (paired helical filaments), which manifest as neurofibrillary tangles.

For example, the cells or animals can comprise a human tau coding sequence (e.g., a genomically integrated human tau coding sequence) comprising a tauopathy-associated mutation. In a specific example, the human tau coding sequence is genomically integrated in the cell or animal. For example, the endogenous Mapt gene can be humanized. An exemplary human tau protein and human MAPT gene are assigned UniProt accession number P10636 and NCBI GeneID 4137, respectively. An exemplary mouse tau protein and mouse Mapt gene are assigned UniProt accession number P10637 and NCBI GeneID 17762, respectively. An exemplary rat tau protein and rat Mapt gene are assigned UniProt accession number P19332 and NCBI GeneID 29477, respectively. The tau protein can comprise a tauopathy-associated mutation (e.g., a tau pathogenic mutation). There are several tau pathogenic mutations, such as pro-aggregation mutations, that are associated with (e.g., segregate with) or cause a tauopathy. Pathogenic tau mutations, which can be either exonic or intronic, generally alter the relative production of tau isoforms and can lead to changes in microtubule assembly and/or the propensity of tau to aggregate. As one example, such a mutation can be an aggregation-sensitizing mutation that sensitizes tau to seeding but does not result in tau readily aggregating on its own. For example, the mutation can be the disease-associated P301S mutation. By P301S mutation is meant the human tau P301S mutation or a corresponding mutation in another tau protein when optimally aligned with the human tau protein. Other pathogenic tau mutations include, for example, A152T, G272V, K280del, P301L, S320F, V337M, R406W, P301L/V337M, K280del/I227P/1308P, G272V/P301L/R406W, and A152T/P301L/S320F. See alzforum.org/mutations/mapt, Brandt et al. (2005) Biochim. Biophys. Acta 1739:331-354, and Wolfe (2009) J. Biol. Chem. 284 (10): 6021-6025, each of which is herein incorporated by reference in its entirety for all purposes. For example, the animal can be a PS19 (Tau P301S (Line PS19); PS19Tg; B6; C3-Tg (Prnp-MAPT*P301S) PS19Vle/J) mouse. The genetic background of this strain is C57BL/6×C3H. PS19 transgenic mice express mutant human microtubule-associated protein tau, MAPT, driven by the mouse prion protein (Prnp) promoter. The transgene encodes the disease-associated P301S mutation and includes four microtubule-binding domains and one N-terminal insert (4R/IN). The transgene inserted at Chr3: 140354280-140603283 (Build GRCm38/mm10), causing a 249 kb deletion that does not affect any known genes. See Goodwin et al. (2019) Genome Res. 29 (3): 494-505, herein incorporated by reference in its entirety for all purposes. Expression of the mutant human tau is five-fold higher than that of the endogenous mouse protein. See Yoshiyama et al. (2007) Neuron 53 (3): 337-351, herein incorporated by reference in its entirety for all purposes. PS19 mice develop neuronal loss and brain atrophy by eight months of age. They also develop widespread tau aggregates, known as neurofibrillary tangle-like inclusions, in the neocortex, amygdala, hippocampus, brain stem, and spinal cord. See Yoshiyama et al. (2007). Prior to the appearance of overt tau pathology by histological methods, the brains of these mice were shown to display tau seeding activity. That is, tau aggregates present in brain homogenate can elicit further tau aggregation, presumably via a prion-like mechanism. See Holmes (2014) Proc. Natl. Acad. Sci. U.S.A. 111 (41): E4376-E4385, herein incorporated by reference in its entirety for all purposes.

Some cells or non-human animals can comprise a humanized MAPT locus. For example, the humanized MAPT locus can comprise an in-locus replacement of non-human animal (e.g., mouse or rat) Mapt sequence (e.g., coding sequence) with corresponding human MAPT sequence (e.g., coding sequence) such that a fully human tau protein is encoded. The cell or animal can be, in some cases, homozygous for the humanized MAPT gene. In other cases, the cell or animal can be heterozygous for the humanized MAPT gene. The animal can comprise the humanized MAPT gene in its germline. The humanized MAPT gene can comprise a human MAPT nucleic acid encoding a human tau protein (e.g., a fully human tau protein) or encoding a chimeric non-human animal/human tau protein. The human MAPT nucleic acid can be a genomic nucleic acid including both coding sequence and non-coding sequence (e.g., introns), or can include only coding sequence. The human MAPT nucleic acid can be a genomic nucleic acid, such as a genomic nucleic acid comprising a region of a human MAPT gene from the start codon to the stop codon (i.e., including introns), or can comprise a human MAPT complementary DNA (cDNA). The human MAPT nucleic acid can be inserted into the non-human animal Mapt genomic locus, or it can replace a corresponding region of the non-human animal Mapt locus (e.g., a region of a human MAPT gene from the start codon to the stop codon can replace a region of the non-human animal Mapt gene from the start codon to the stop codon). The humanized MAPT gene (or the human MAPT nucleic acid) can be operably linked to the endogenous non-human animal Mapt promoter. In other words, expression of the humanized MAPT gene can be driven by the endogenous non-human animal Mapt promoter.

In another example, the cells or animals can express an apolipoprotein E (ApoE) protein (e.g., a human ApoE protein), such as an ApoE3 or ApoE4 protein (e.g., a human ApoE3 or human ApoE4 protein). For example, the cells or animals can express the ApoE protein in neurons. In some cells or animals, only a single ApoE allele is expressed. In some embodiments, only ApoE4 (e.g., human ApoE4) is expressed. In some embodiments, only ApoE3 (e.g., human ApoE3) is expressed. ApoE, a major component of LDL and VLDL lipoproteins circulating in blood, mediates uptake of lipoproteins into cells by binding to ApoE receptors, principally the LDL-receptor. ApoE is also highly expressed in brain, primarily in astrocytes. Human ApoE is primarily expressed in three isoforms (ApoE2, ApoE3, and ApoE4) that differ only by two residues. ApoE4 constitutes the most important genetic risk factor for AD, ApoE3 is neutral, and ApoE2 is protective. ApoE4 is associated with increased Aβ-accumulation in brain.

For example, the cells or animals can comprise a human ApoE coding sequence (e.g., a genomically integrated human ApoE coding sequence). In a specific example, the human ApoE coding sequence is genomically integrated in the cell or animal. In another specific example, the endogenous ApoE gene can be humanized. An exemplary human ApoE protein and human APOE gene are assigned UniProt accession number P02649 and NCBI GeneID 348, respectively. An exemplary mouse ApoE protein and mouse ApoE gene are assigned UniProt accession number P08226 and NCBI GeneID 11816, respectively. An exemplary rat ApoE protein and rat ApoE gene are assigned UniProt accession number P02650 and NCBI GeneID 25728, respectively.

Some cells or non-human animals can comprise a humanized APOE locus. For example, the humanized APOE locus can comprise an in-locus replacement of non-human animal (e.g., mouse or rat) Apoe sequence (e.g., coding sequence) with corresponding human APOE sequence (e.g., coding sequence) such that a fully human ApoE protein is encoded. The cell or animal can be, in some cases, homozygous for the humanized APOE gene. In other cases, the cell or animal can be heterozygous for the humanized APOE gene. The animal can comprise the humanized APOE gene in its germline. The humanized APOE gene can comprise a human APOE nucleic acid encoding a human ApoE protein (e.g., a fully human ApoE protein) or a chimeric non-human animal/animal ApoE protein. In one example, the humanized APOE gene can comprise a human APOE nucleic acid encoding a human ApoE4 protein (e.g., a fully human ApoE4 protein). In another example, the humanized APOE gene can comprise a human APOE nucleic acid encoding a human ApoE3 protein (e.g., a fully human ApoE3 protein). The human APOE nucleic acid can be a genomic nucleic acid including both coding sequence and non-coding sequence (e.g., introns), or can include only coding sequence. The human APOE nucleic acid can be a genomic nucleic acid, such as a genomic nucleic acid comprising a region of a human APOE gene from the start codon to the stop codon (i.e., including introns), or can comprise a human APOE complementary DNA (cDNA). The human APOE nucleic acid can be inserted into the non-human animal Apoe genomic locus, or it can replace a corresponding region of the non-human animal Apoe locus (e.g., a region of a human APOE gene from the start codon to the stop codon can replace a region of the non-human animal Apoe gene from the start codon to the stop codon). The humanized APOE gene (or the human APOE nucleic acid) can be operably linked to the endogenous non-human animal Apoe promoter. In other words, expression of the humanized APOE gene can be driven by the endogenous non-human animal Apoe promoter.

In another example, the cells or animals can comprise a genomically integrated human amyloid-beta precursor protein (APP) coding sequence comprising one or more mutations associated with AD (e.g., FAD). For example, the endogenous App gene can be humanized. APP is described in more detail above. An exemplary human APP protein and human APP gene are assigned UniProt accession number P05067 and NCBI GeneID 351, respectively. An exemplary mouse APP protein and mouse App gene are assigned UniProt accession number P12023 and NCBI GeneID 11820, respectively. An exemplary rat APP protein and rat App gene are assigned UniProt accession number P08592 and NCBI GeneID 54226, respectively. The amyloid-beta precursor proteins described herein can comprise one or more mutations associated with AD (e.g., FAD). Mutations in amyloid-beta precursor protein are associated with familial forms of early onset AD as well as with Cerebral Amyloid Angiopathy (CAA). Pathogenic mutations generally alter processing by secretases, leading in an overall increase in Aβ production and/or a change in the ratio of specific Aβ peptides. See alzforum.org/mutations/app, TCW et al. (2017) Cold Spring Harb. Perspect Med. 7 (6): a024539, and Dai et al. (2018) Oncotarget 9:15132-15143, each of which is herein incorporated by reference in its entirety for all purposes. In a specific example, an amyloid-beta precursor protein described herein comprises a K670N/M671L mutation (with reference to the canonical human APP770 isoform, or K595N/M596L mutation with reference to the APP695 isoform), which is also known as the Swedish mutation. By K670N/M671L mutation is meant a K670N/M671L mutation in the canonical human APP770 isoform or a corresponding mutation in another amyloid-beta precursor protein when optimally aligned with the canonical human amyloid-beta precursor protein (e.g., a K595N/M596L mutation in the human APP695 isoform). The Swedish mutation (K670N/M671L) repeatedly has been shown to increase total Aβ levels by facilitating amyloid-beta precursor protein cleavage by BACE1. In carriers of the Swedish mutation, both Aβ40 and Aβ42 are elevated in the plasma. See, e.g., Shin et al. (2010) BMB Rep. 43 (10:704-709, herein incorporated by reference in its entirety for all purposes.

Some cells or non-human animals can comprise a humanized APP locus. The cell or animal can be, in some cases, homozygous for the humanized APP gene. In other cases, the cell or animal can be heterozygous for the humanized APP gene. The animal can comprise the humanized APP gene in its germline. The humanized APP gene can comprise a human APP nucleic acid encoding a human APP protein (e.g., a fully human APP protein) or a chimeric non-human animal/human APP protein. The human APP nucleic acid can be a genomic nucleic acid including both coding sequence and non-coding sequence (e.g., introns), or can include only coding sequence. The human APP nucleic acid can be a genomic nucleic acid, such as a genomic nucleic acid comprising a region of a human APP gene from the start codon to the stop codon (e.g., including introns), or can comprise a human APP complementary DNA (cDNA). The human APP nucleic acid can be inserted into the non-human animal App genomic locus, or it can replace a corresponding region of the non-human animal App locus (e.g., a region of a human APP gene from the start codon to the stop codon can replace a region of the non-human animal App gene from the start codon to the stop codon). The humanized APP gene (or the human APP nucleic acid) can be operably linked to the endogenous non-human animal App promoter. In other words, expression of the humanized APP gene can be driven by the endogenous non-human animal App promoter.

In some embodiments, the cell or animal comprises a humanized MAPT locus (e.g., expressing a human tau protein), a humanized APOE locus (e.g., expressing only human ApoE4 or only human ApoE3), and a humanized APP locus (e.g., expressing an APP protein such as a chimeric non-human animal/human APP protein with Swedish mutations).

### III. Methods of Making Non-Human Animal Models of Alzheimer's Disease and Methods of Modeling Alzheimer's Disease

The animals (e.g., non-human animals) described herein can be made by any suitable means. Such animals are useful for modeling Alzheimer's disease (AD). The animals can be made, for example, by administering a composition described herein (i.e., an amyloid-beta precursor protein and/or presenilin-1 disclosed herein, such as in the form of nucleic acids, nucleic acid constructs, expression constructs, vectors, or lipid nanoparticles disclosed herein) to the animal, such as any of the animals described above in Section II.E. Likewise, AD can be modeled in an animal, for example, by administering a composition described herein (i.e., an amyloid-beta precursor protein and/or presenilin-1 disclosed herein, such as in the form of nucleic acids, nucleic acid constructs, expression constructs, vectors, or lipid nanoparticles disclosed herein) to any of the animals described herein.

The composition (i.e., an amyloid-beta precursor protein and/or presenilin-1 disclosed herein, such as in the form of nucleic acids, nucleic acid constructs, expression constructs, vectors, or lipid nanoparticles disclosed herein) can be administered to the animal in any suitable form and by any suitable route of administration.

Administration in vivo can be by any suitable route such that the composition reaches the intended target cell(s) (e.g., neurons in the brain of the subject and/or glial cells in the brain of the subject) or target tissue (e.g., brain). Examples of routes of administration include parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. For example, the composition may be administered directly to the brain of a subject or to neurons in the brain of a subject. In a specific example, administration to a subject is by intrathecal injection or by intracranial injection (e.g., stereotactic surgery for injection in the hippocampus and other brain regions, or intracerebroventricular injection). In a specific example, administration to a subject is by intracerebroventricular injection. In another specific example, administration to a subject is by intracranial injection. In another specific example, administration to a subject is by intrathecal injection. In another specific example, administration to a subject is by intrahippocampal injection.

The frequency of administration and the number of dosages can depend on the half-life of the composition being administered and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

In a specific example, the composition (i.e., an amyloid-beta precursor protein and/or presenilin-1 disclosed herein, such as in the form of nucleic acids, nucleic acid constructs, expression constructs, vectors, or lipid nanoparticles disclosed herein) can be administered to the brain of the animal (e.g., via intrahippocampal injection, intracerebroventricular injection, intracranial injection, intrathecal injection, or stereotactic injection).

In some methods, the amyloid-beta precursor protein and presenilin-1 (such as in the form of nucleic acids, nucleic acid constructs, expression constructs, vectors, or lipid nanoparticles disclosed herein) can be administered together (e.g., simultaneously or sequentially in any order). In some methods, the amyloid-beta precursor protein and presenilin-1 (such as in the form of nucleic acids, nucleic acid constructs, expression constructs, vectors, or lipid nanoparticles disclosed herein) can be administered simultaneously or not simultaneously. For example, in a method comprising administering a composition comprising the amyloid-beta precursor protein and the presenilin-1 (such as in the form of nucleic acids, nucleic acid constructs, expression constructs, vectors, or lipid nanoparticles disclosed herein), they can be administered separately. For example, the amyloid-beta precursor protein (such as in the form of nucleic acids, nucleic acid constructs, expression constructs, vectors, or lipid nanoparticles disclosed herein) can be administered prior to, subsequent to, or at the same time as the presenilin-1 (such as in the form of nucleic acids, nucleic acid constructs, expression constructs, vectors, or lipid nanoparticles disclosed herein).

Such animals can develop one or more symptoms or phenotypes of AD following administration of the composition. For example, the animal can develop one or more or all of the following: Aβ plaque pathology; loss of synapses; gliosis; dystrophic neurites around Aβ plaques; and intracellular lysosome aggregation around Aβ plaques. As another example, the animal can develop one or more or all of the following: tau aggregates in dystrophic neurites surrounding Aβ plaques (NP-tau); neurofibrillary tangles (NFTs); and increased phosphorylated tau in the somatodendritic compartment of neurons. As another example, the animal can develop the following: co-deposition of ApoE (e.g., human ApoE) protein in Aβ plaques. These phenotypes can be increased, for example, compared to a control animal that has not been administered the composition. In one example, the animal can develop these symptoms or phenotypes within 1 month after administration. In another example, the animal can develop these symptoms or phenotypes within 2 months after administration. In another example, the animal can develop these symptoms or phenotypes within 3 months after administration. In another example, the animal can develop these symptoms or phenotypes within 4 months after administration. In another example, the animal can initially develop these symptoms or phenotypes no more than 6 months after administration. In another example, the animal can initially develop these symptoms or phenotypes no more than 5 months after administration. In another example, the animal can initially develop these symptoms or phenotypes no more than 4 months after administration. In another example, the animal can initially develop these symptoms or phenotypes no more than 3 months after administration. In another example, the animal can initially develop these symptoms or phenotypes no more than 2 months after administration. In another example, the animal can initially develop these symptoms or phenotypes no more than 1 month after administration. In a specific example, the animal develops Aβ plaque pathology (i.e., Aβ plaques) within 1 month after administration.

### IV. Methods of Testing Candidate Therapeutic Agents for Treating or Preventing Alzheimer's Disease

The animals (e.g., non-human animals) disclosed herein can be used in methods of assessing a therapeutic candidate for the treatment of Alzheimer's disease (AD) or the amelioration of a symptom or phenotype of AD. For example, such methods can comprise administering a candidate agent to the animal, performing one or more assays to determine if the candidate agent has an effect on an AD phenotype, and identifying the candidate agent that ameliorates the AD phenotype as a therapeutic candidate. Such symptoms and phenotypes, and methods of assessing them, are described in more detail elsewhere herein.

The assessing can be compared to a control. For example, the assessing can be compared to the animal before administration of the candidate agent or can be compared to a control animal that has not been administered the candidate agent.

A candidate agent can be any reagent, such a known therapeutic agent for AD or a putative therapeutic agent for AD, or a reagent being screened for activity in treating or preventing AD. The candidate agent can be, for example, an antibody or antigen-binding protein or any other large molecule or small molecule that targets a protein, an RNA, a gene, or any other target. Alternatively, the candidate agent can be any biological or chemical agent that targets a protein, an RNA, a gene, or any other target.

For example, a candidate agent can be an antigen-binding protein, such as an antigen-binding protein targeting a protein or antigen associated with AD (e.g., a pathogenic protein). The term “antigen-binding protein” includes any protein that binds to an antigen. Examples of antigen-binding proteins include an antibody, an antigen-binding fragment of an antibody, a multispecific antibody (e.g., a bi-specific antibody), an scFv, a bis-scFv, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

Other candidate agents include small molecules, such as a small molecule targeting a cell, protein, RNA, gene, or any other target associated with AD (e.g., a pathogenic protein).

Other candidate agents can include genome editing reagents such as a nuclease agent (e.g., a Clustered Regularly Interspersed Short Palindromic Repeats (CRISPR)/CRISPR-associated (Cas) (CRISPR/Cas) nuclease, a zinc finger nuclease (ZFN), or a Transcription Activator-Like Effector Nuclease (TALEN)) that cleaves a recognition site within a target gene, such as a gene associated with AD (e.g., a gene encoding a pathogenic protein). Likewise, a candidate agent can be an exogenous donor nucleic acid (e.g., a targeting vector or single-stranded oligodeoxynucleotide (ssODN)) designed to recombine with such a target gene.

Other candidate agents can include RNAi agents designed to target RNAs (e.g., messenger RNAs), such as RNAs associated with AD (e.g., an mRNA encoding a pathogenic protein). An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to, short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Other candidate agents can include antisense oligonucleotides (ASOs) designed to target a target RNA, such as RNAs associated with AD (e.g., an RNA encoding a pathogenic protein). Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g., LNA or 2′-MOE) on each terminus flanking a central 8-10 base gap of DNA. After binding the target RNA, the DNA-RNA hybrid acts substrate for RNase H.

The candidate agent can be administered to the animal by any suitable route of administration. For example, the candidate agent can be administered to the brain of the animal (e.g., via intrahippocampal injection, intracerebroventricular injection, intracranial injection, intrathecal injection, or stereotactic injection).

Likewise, the animals (e.g., non-human animals) disclosed herein can be used in methods of assessing a therapeutic candidate for the prevention of AD or the prevention of a symptom or phenotype of AD. For example, such methods can comprise administering a candidate agent to an animal, administering a composition described herein (i.e., an amyloid-beta precursor protein and/or presenilin-1 disclosed herein, such as in the form of nucleic acids, nucleic acid constructs, expression constructs, vectors, or lipid nanoparticles disclosed herein) to the animal, performing one or more assays to determine if the candidate agent has an effect on an AD phenotype, and identifying the candidate agent that prevents the AD phenotype as a therapeutic candidate. Such symptoms and phenotypes, and methods of assessing them, are described in more detail elsewhere herein.

The assessing can be compared to a control. For example, the assessing can be compared to a control animal that has been administered the composition (i.e., an amyloid-beta precursor protein and/or presenilin-1 disclosed herein, such as in the form of nucleic acids, nucleic acid constructs, expression constructs, vectors, or lipid nanoparticles disclosed herein) but has not been administered the candidate agent.

The composition (i.e., an amyloid-beta precursor protein and/or presenilin-1 disclosed herein, such as in the form of nucleic acids, nucleic acid constructs, expression constructs, vectors, or lipid nanoparticles disclosed herein) can be administered to the animal in any suitable form and by any suitable route of administration, as disclosed in more detail elsewhere herein.

In an example, the composition (i.e., an amyloid-beta precursor protein and/or presenilin-1 disclosed herein, such as in the form of nucleic acids, nucleic acid constructs, expression constructs, vectors, or lipid nanoparticles disclosed herein) can be administered to the brain of the animal (e.g., via intrahippocampal injection, intracerebroventricular injection, intracranial injection, intrathecal injection, or stereotactic injection). Similarly, the candidate agent can be administered to the animal by any suitable route of administration. For example, the candidate agent can be administered to the brain of the animal (e.g., via intrahippocampal injection, intracerebroventricular injection, intracranial injection, intrathecal injection, or stereotactic injection).

In some methods, the amyloid-beta precursor protein and presenilin-1 (such as in the form of nucleic acids, nucleic acid constructs, expression constructs, vectors, or lipid nanoparticles disclosed herein) can be administered together (e.g., simultaneously or sequentially in any order). In some methods, the amyloid-beta precursor protein and presenilin-1 (such as in the form of nucleic acids, nucleic acid constructs, expression constructs, vectors, or lipid nanoparticles disclosed herein) can be administered simultaneously or not simultaneously. For example, in a method comprising administering a composition comprising the amyloid-beta precursor protein and the presenilin-1 (such as in the form of nucleic acids, nucleic acid constructs, expression constructs, vectors, or lipid nanoparticles disclosed herein), they can be administered separately. For example, the amyloid-beta precursor protein (such as in the form of nucleic acids, nucleic acid constructs, expression constructs, vectors, or lipid nanoparticles disclosed herein) can be administered prior to, subsequent to, or at the same time as the presenilin-1 (such as in the form of nucleic acids, nucleic acid constructs, expression constructs, vectors, or lipid nanoparticles disclosed herein).

The assessing can be any suitable time after administration of the composition (i.e., an amyloid-beta precursor protein and/or presenilin-1 disclosed herein, such as in the form of nucleic acids, nucleic acid constructs, expression constructs, vectors, or lipid nanoparticles disclosed herein). In one example, the assessing can be at least 1 month after administration. In another example, the assessing can be at least 2 months after administration. In another example, the assessing can be at least 3 months after administration. In another example, the assessing can be at least 4 months after administration. In one example, the assessing can be within 1 month after administration. In another example, the assessing can be within 2 months after administration. In another example, the assessing can be within 3 months after administration. In another example, the assessing can be within 4 months after administration. In another example, the assessing may be no more than 6 months after administration. In another example, the assessing may be no more than 5 months after administration. In another example, the assessing may be no more than 4 months after administration. In another example, the assessing may be no more than 3 months after administration. In another example, the assessing may be no more than 2 months after administration. In another example, the assessing may be no more than 1 month after administration.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Brief Description of the Sequences

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

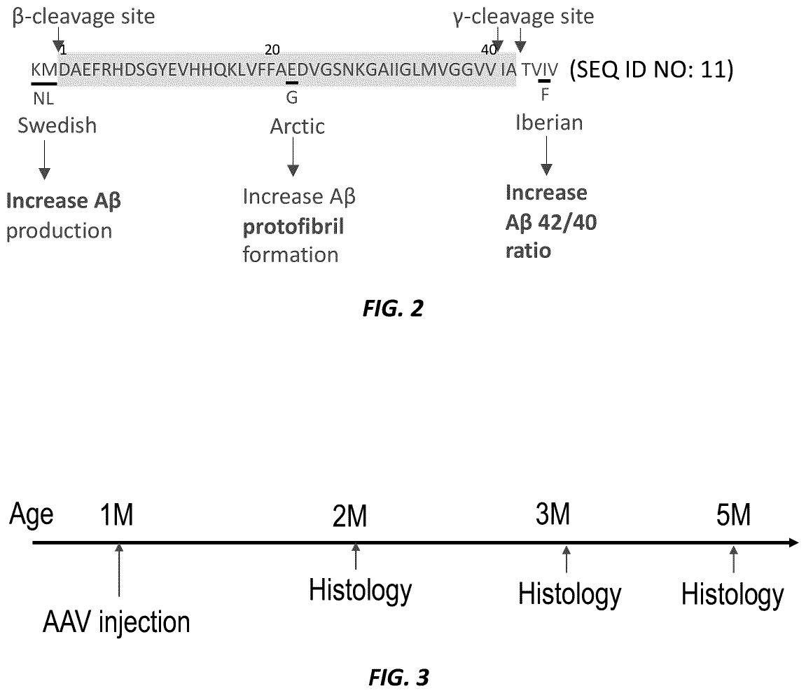

TABLE 2
Description of Sequences.

| SEQ ID NO | Type | Description |
|---|---|---|
| 1 | Protein | Human APP695 isoform with Swedish [KM595/596NL], Arctic [E618G], and |
| | | Iberian [I641F] FAD mutations |
| 2 | DNA | CDS for human APP695 isoform with Swedish [KM595/596NL], Arctic [E618G], |
| | | and Iberian [I641F] FAD mutations |
| 3 | Protein | Human PSEN1 with M146L and L286V FAD mutations |
| 4 | DNA | CDS for human PSEN1 with M146L and L286V FAD mutations |
| 5 | Protein | Human APP695 (P05067-4; NP_958817.1) |
| 6 | DNA | CDS for human APP695 (CCDS13577.1) |
| 7 | Protein | Human APP770 (P05067-1; NP_000475.1) |
| 8 | DNA | CDS for human APP770 (CCDS13576.1) |
| 9 | Protein | Human PSEN1 (P-49768-1; NP_000012.1) |
| 10 | DNA | CDS for human PSEN1 (CCDS9812.1) |
| 11 | Protein | Peptide from FIG. 2 |

### EXAMPLES

### Example 1. Mouse Model Using AAV Delivery of Human APP and PSEN1 with FAD Mutations

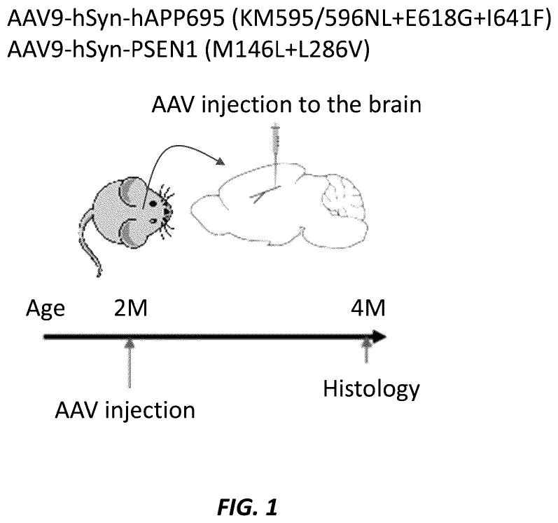

To address the limitations of current transgenic models of Alzheimer's disease, which are time-consuming to generate and take extended periods of time for phenotypes to develop, a new model was developed by injecting AAV vectors together directly into the brain of wild type mice at ˜2 months of age. A rapid onset of Alzheimer's pathology was observed, providing an advantage over existing transgenic models. See FIG. 1.

Two AAV vectors were generated with human familial Alzheimer's disease (FAD) mutant forms of amyloid-beta precursor protein (APP) and presenilin-1 (PSEN1). Both were recombinant AAV9 vectors driven by a human synapsin-1 promoter. One vector was generated for the expression of the human APP695 isoform with Swedish [KM595/596NL], Arctic [E618G], and Iberian [1641F] FAD mutations (SEQ ID NO: 1, encoded by SEQ ID NO: 2). See FIG. 2. Another vector was generated for the expression of human PSEN1 with M146L and L286V FAD mutations (SEQ ID NO: 3, encoded by SEQ ID NO: 4).

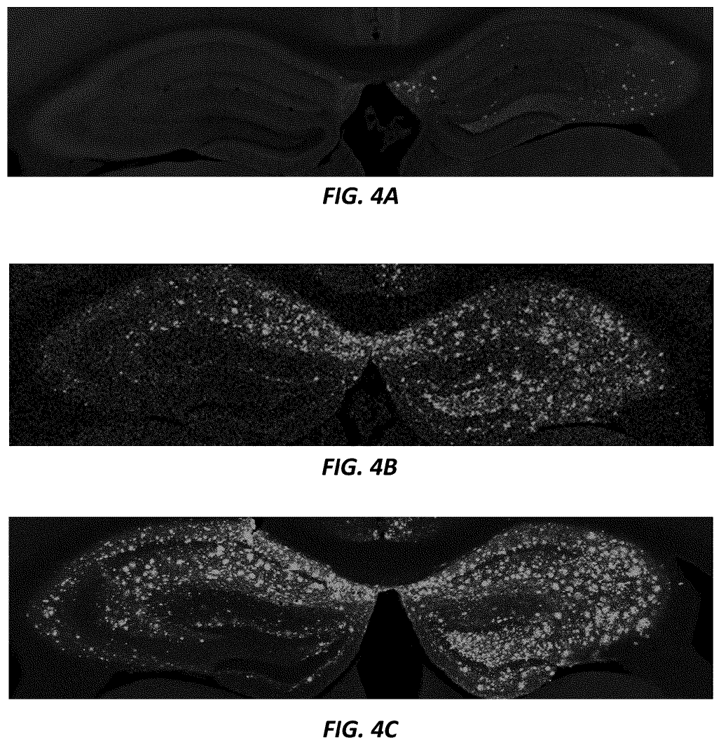

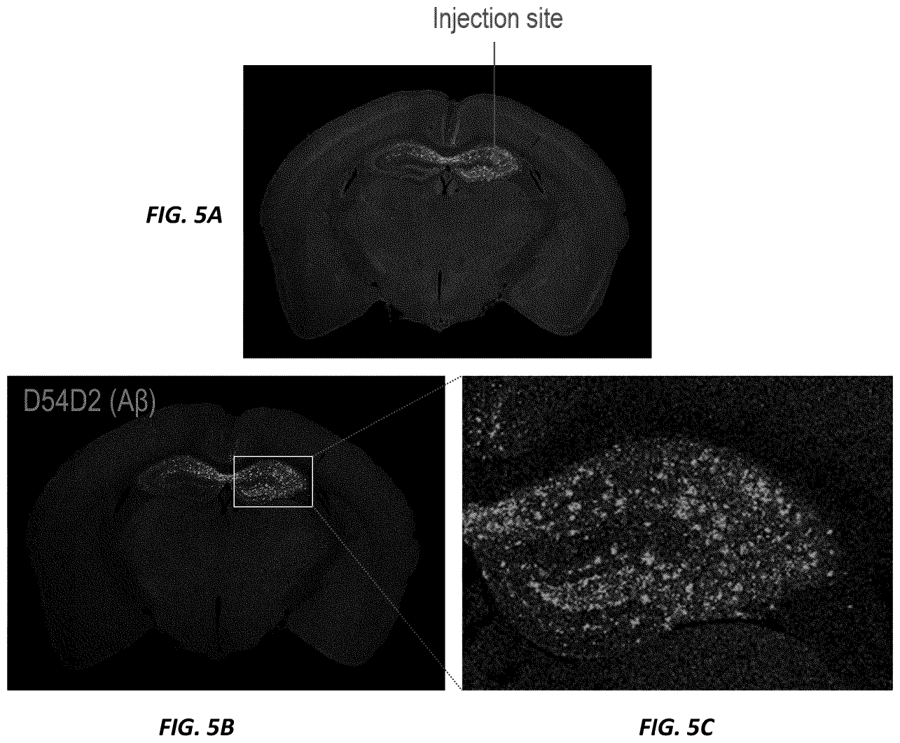

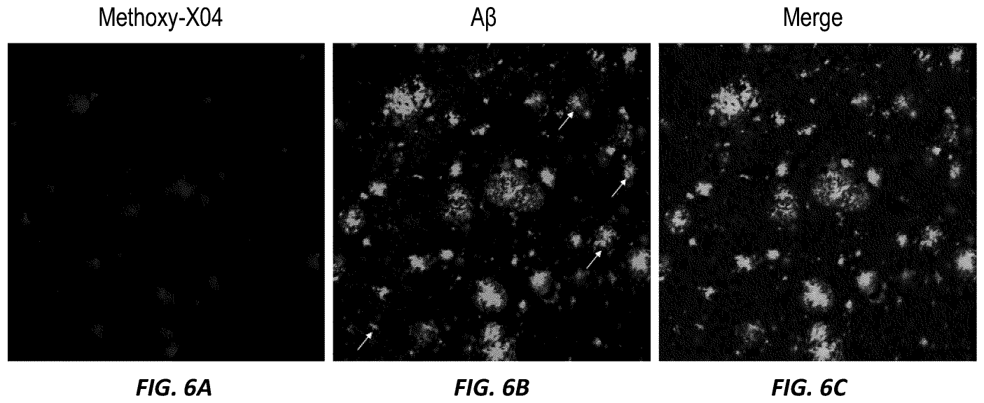

The APP and PSEN1 AAV vectors were injected into the brains of 1-month old female C57BLJ mice. Specifically, 1 μL of each AAV (10¹³ gc/mL) was injected into the hippocampus at coordinates (A/P: −1.8, M/L: 1.5, D/V: −2.0) relative to bregma. Histology was performed on brain tissue of mice at 1-, 2-, and 4-months post injection. See FIG. 3. An age-dependent accumulation of amyloid plaques was observed with increasing amounts of Aβ plaques present from one month (FIG. 4A) to two months (FIG. 4B) to four months (FIG. 4C) post-AAV injection, as shown by staining with a β-amyloid antibody (Cell Signaling D54D2). Within one month post-AAV injection, Aβ plaque pathology was observed. At two months post AAV injection, the Aβ plaque pathology was very robust, as shown by the strong staining near the injection site. See FIGS. 5A-5C. Additionally, staining with methoxy-X04, a fluorescent Aβ probe that binds selectively to fibrillar β-sheet deposits, showed dense core plaques surrounded by filamentous plaques. See FIG. 6A-6C.

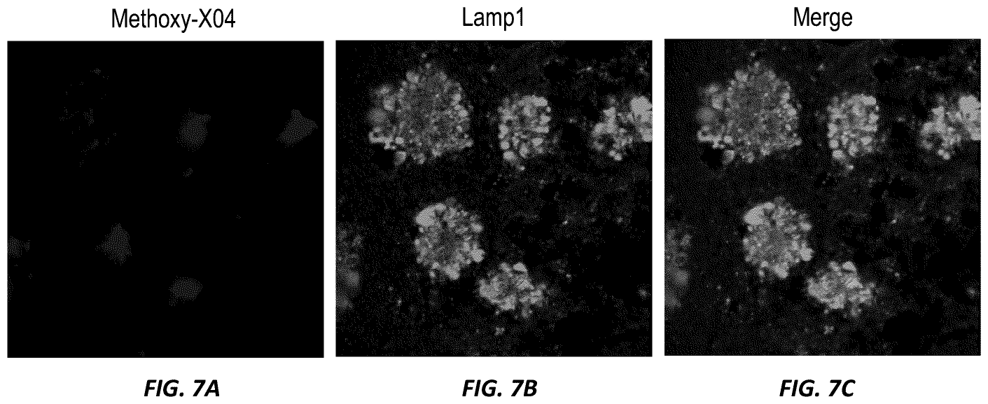

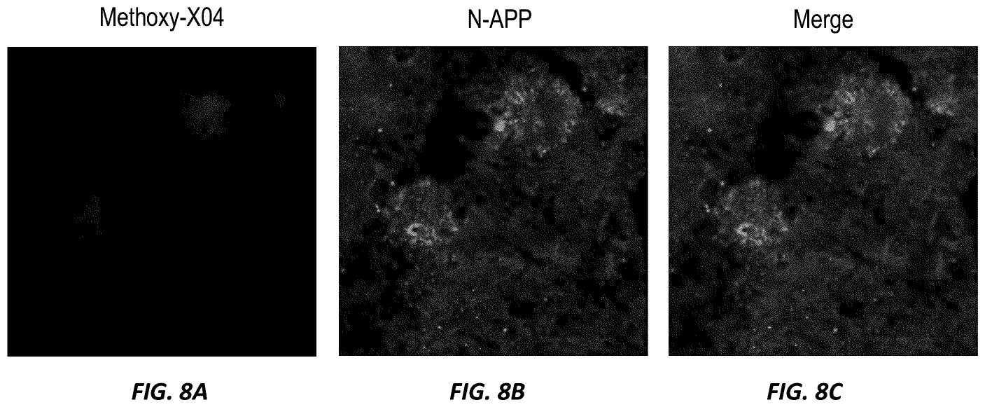

At two months post-injection, dystrophic neurites were also observed around plaques, as evidenced by staining for Lamp1, a lysosomal protein highly enriched in dystrophic neurites. The presence of dystrophic neurites (FIG. 7B) around dense core plaques stained by methoxy-X04 (FIG. 7A) indicates intracellular lysosome aggregation (FIG. 7C), an Alzheimer's disease brain pathology. Staining for N-APP, another marker which is highly enriched in dystrophic neurites was also observed at two months post-AAV injection. See FIG. 8A-8C.

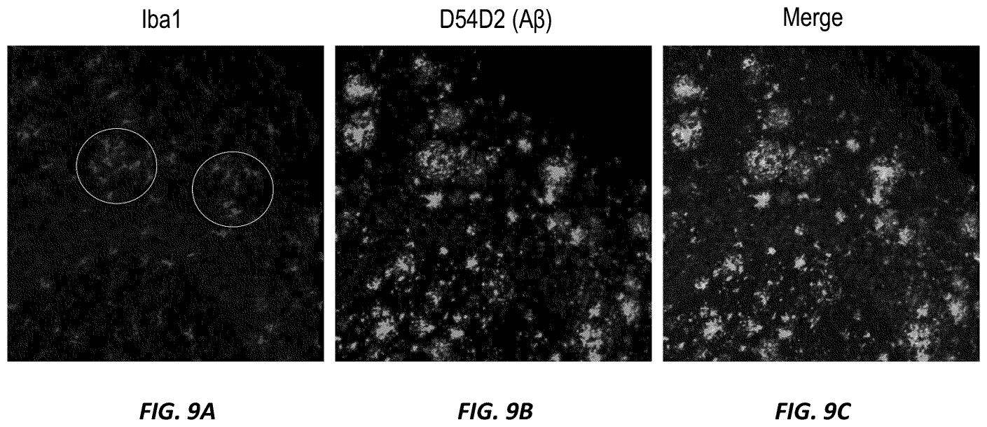

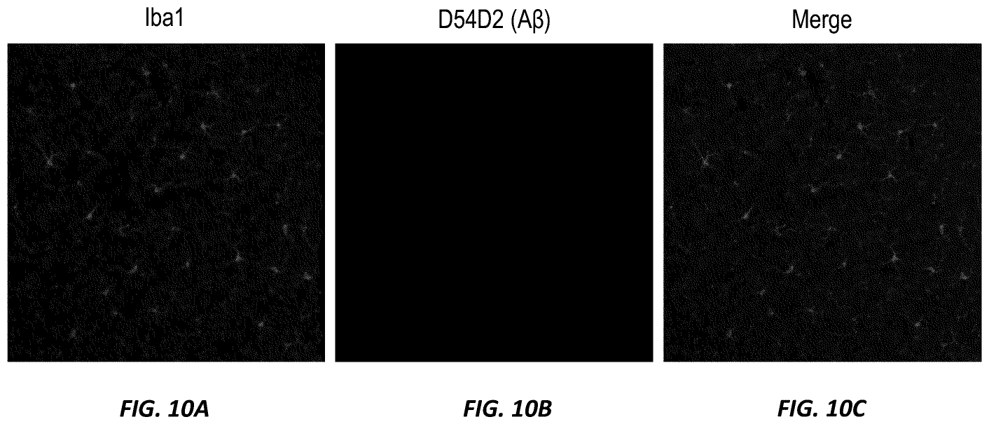

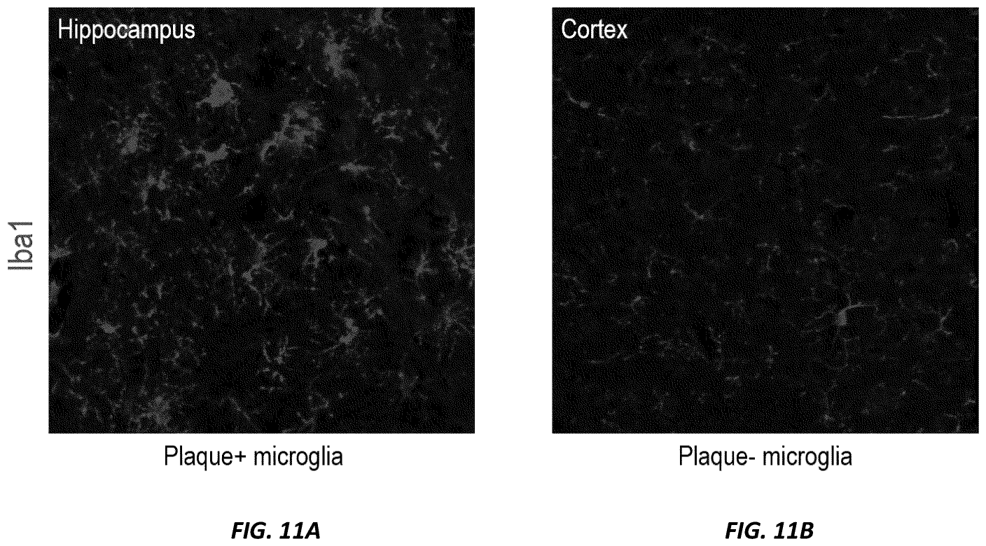

Histological analysis at two months post-AAV injection revealed microglia cluster around amyloid plaques in the hippocampus as evidenced by Iba1 staining, a marker for microglial detection. See FIG. 9A-9C. However, microglia were not observed in the cortex where no plaques developed. See FIG. 10A-10C. In the hippocampus where plaques were observed, the microglia exhibited morphological changes (FIG. 11A) in comparison to the cortex without plaques (FIG. 11B).

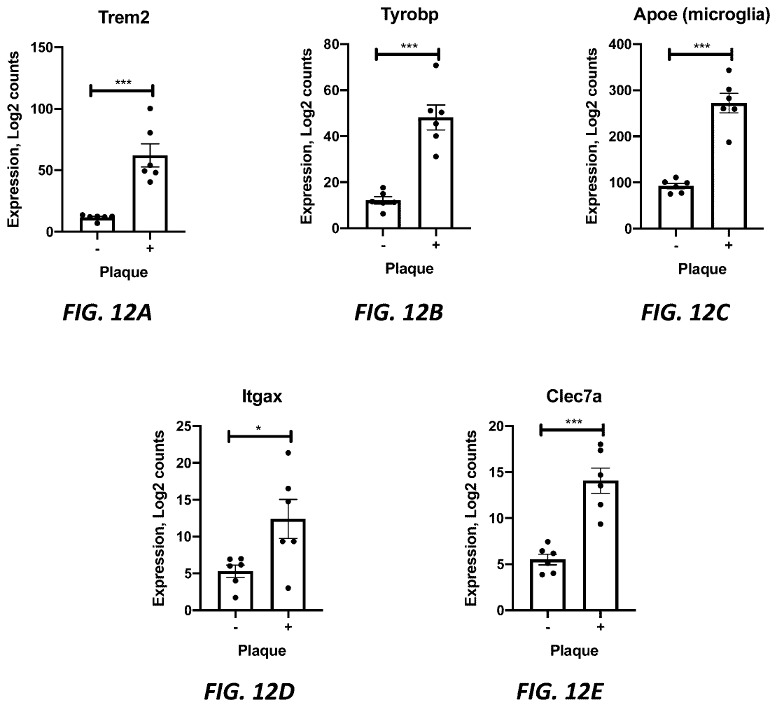

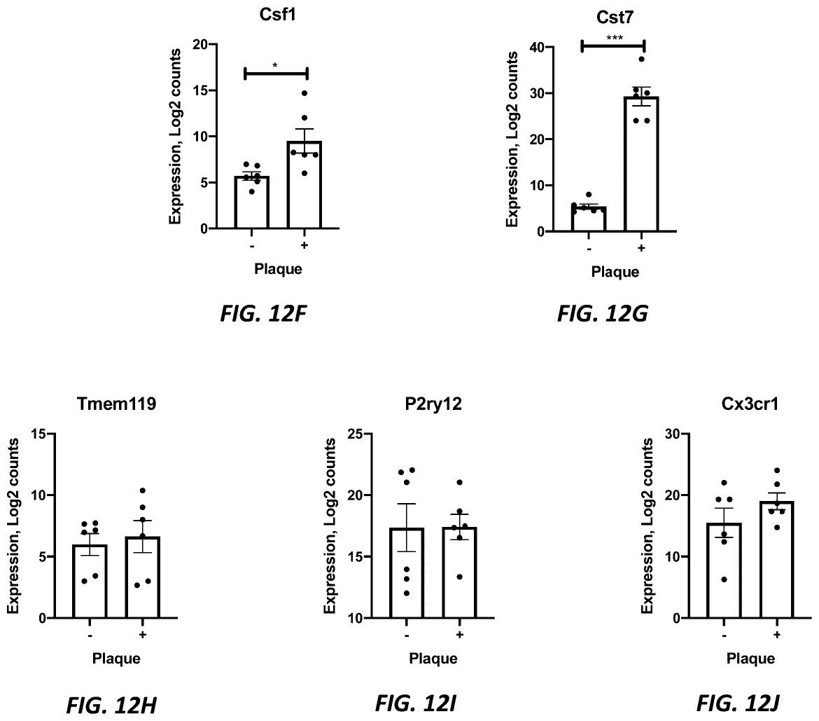

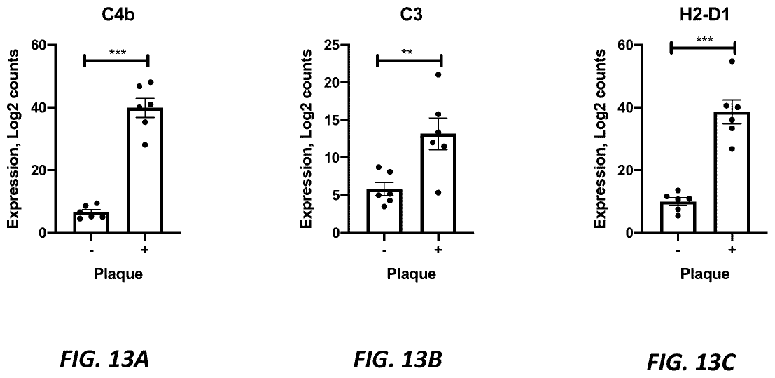

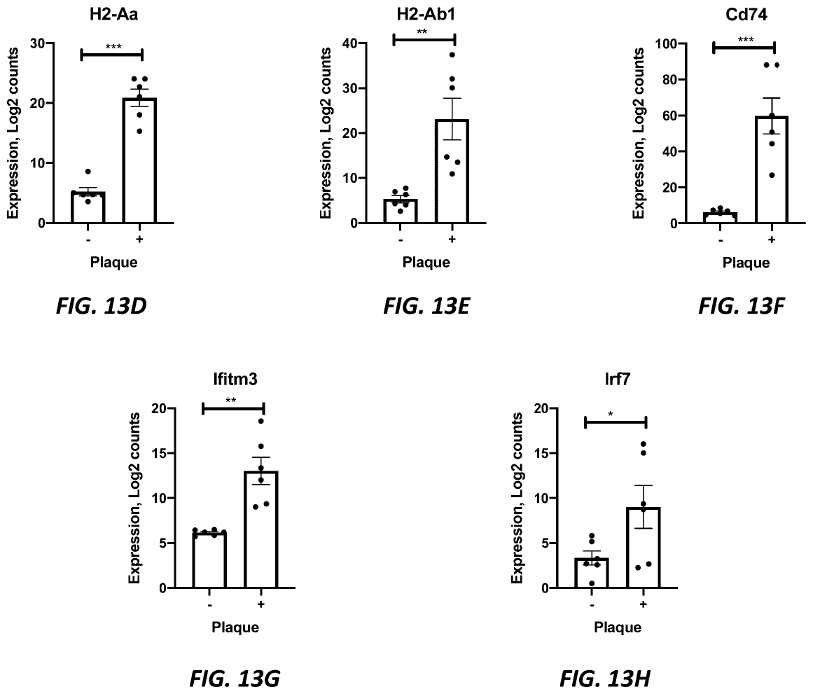

The microglia in hippocampus tissue containing plaques exhibited a disease-associated microglia (DAM) signature consistent with literature reports of microglia associated with Alzheimer's disease. See, e.g., Keren-Shaul et al. (2017) Cell 169 (7): 1276-1290, herein incorporated by reference in its entirety for all purposes. At four months post-AAV injection, expression of Trem2, Tyrobp, Apoc, Itgax, Clec7α, Csf1, and Cst7 was significantly upregulated in microglia in the plaques+ brain regions as assessed via spatial transcriptomics (adding probe to detect mRNA (in situ sequencing); we did sequencing in microglia in amyloid plaque+ area vs in microglia in amyloid plaque-area) . . . . See FIGS. 12A-12G. Upregulation of Tmem119, P2ry12, and Cx3crl was not statistically significant. See FIGS. 12H-12J. The microglia associated with plaques exhibited signatures of late response microglia, which has previously been observed in a neurodegeneration mouse model. See Mathys et al. (2017) Cell Rep. 10; 21 (2): 366-380, herein incorporated by reference in its entirety for all purposes. At four months post-AAV injection, expression of complement components C4b and C3, MHC-I component H2-D1, and MHC-II components H2-Aa, H2-Ab1, and Cd74 all showed significant upregulation in tissue/microglial cells in the plaques+ brain regions. See FIGS. 13A-13F. The plaque+ sample is comparing microglia transcriptome in microglia in the brain regions that exhibit amyloid plaques vs in the brain region without plaques. Additionally, Ifitm3 and Irf7 were significantly upregulated, indicating an interferon response. See FIGS. 13G-13H.

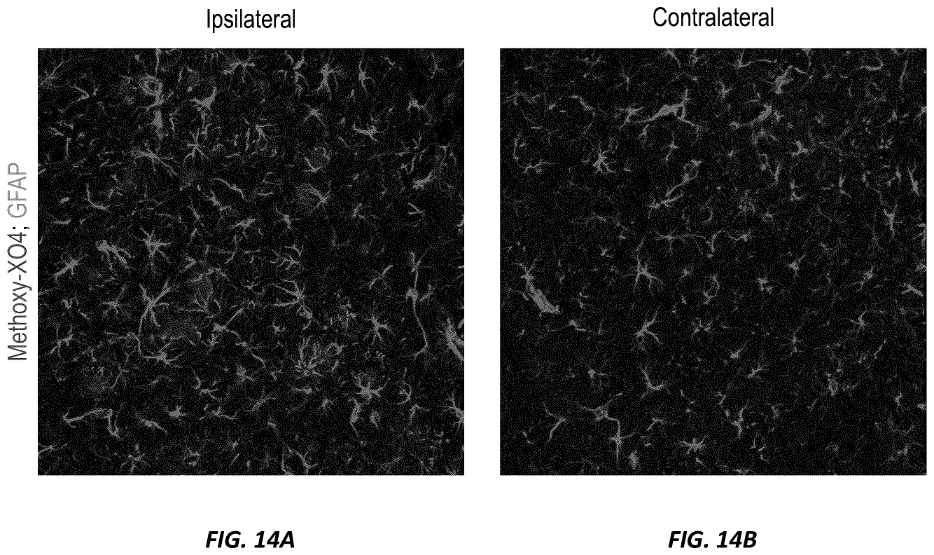

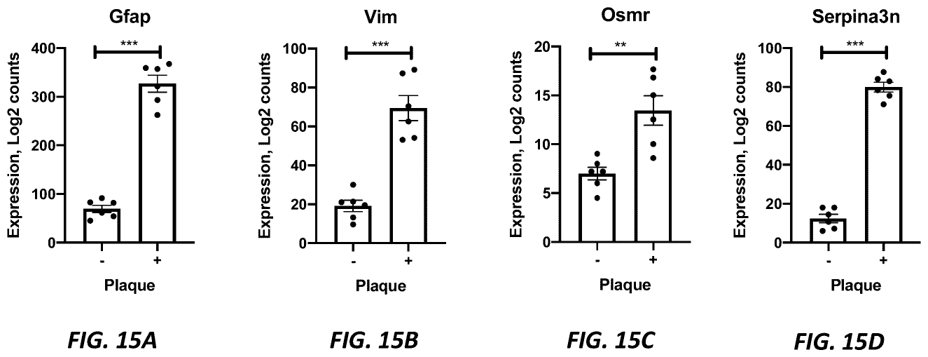

Furthermore, histology at two months post-AAV injection revealed that glial fibrillary acidic protein (GFAP) immunoreactivity is higher in the hippocampus where there are plaques. FIG. 14A shows elevated staining of GFAP in the ipsilateral side of the hippocampus associated with plaques stained by methoxy-X04 in comparison to the contralateral side seen in FIG. 14B. Further data indicate that astrocytes associated with plaques exhibit disease-associated astrocyte (DAA) signatures. Four months post-AAV injection, expression of Gfap, Vim, Osmr, and Serpina3n were significantly elevated in tissue/astrocytes associated with plaques. See FIGS. 15A-15D. These results are consistent with existing transgenic Alzheimer's models, wherein GFAP, VIM, and SerpinA3N were observed in the dentate gyrus of 5×FAD mice.

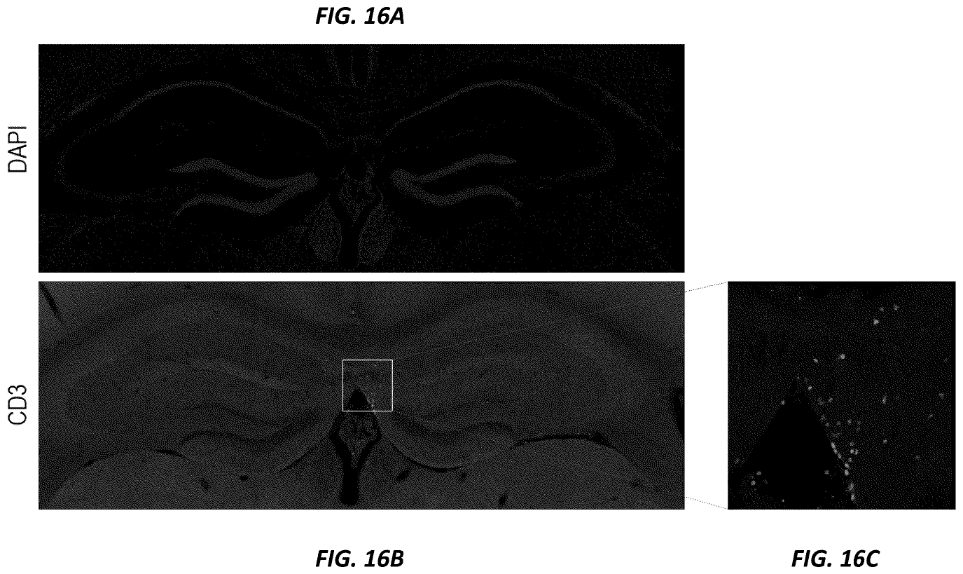

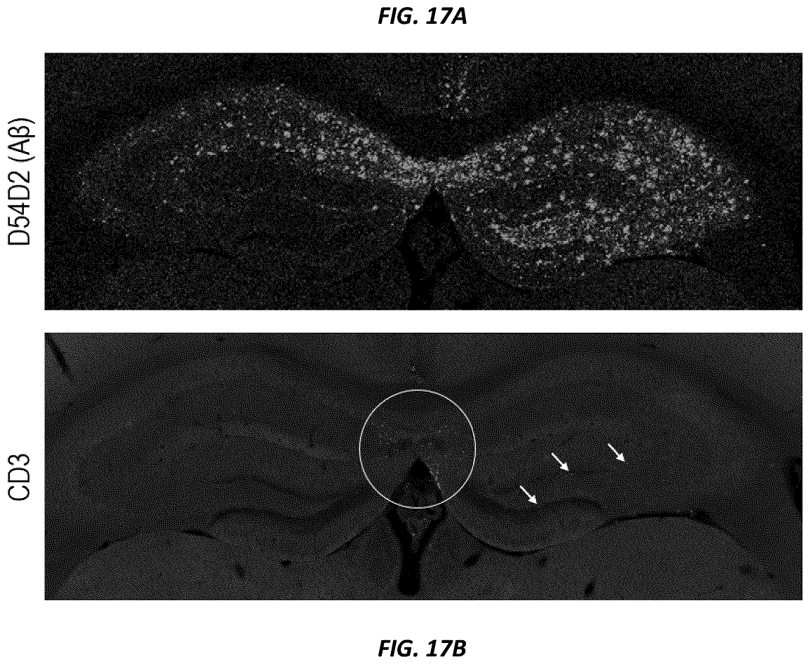

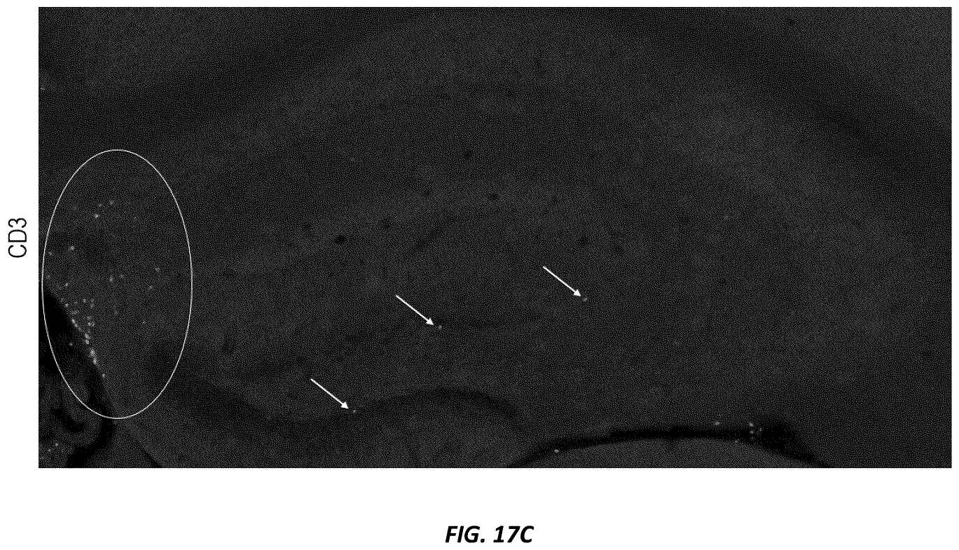

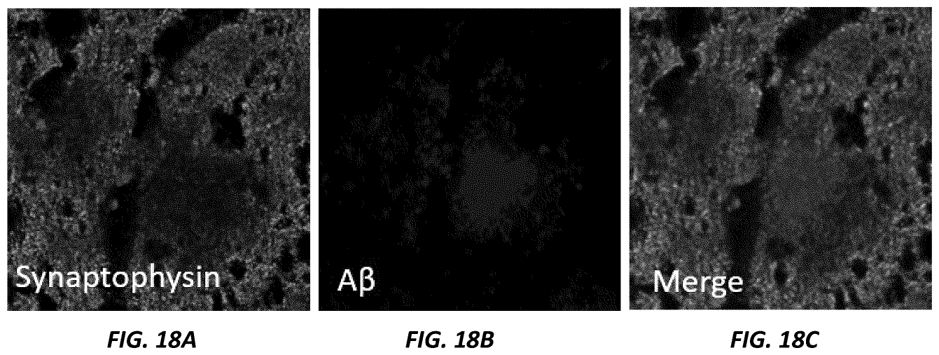

Evidence of T cell infiltration was observed the hippocampus of mice two months post-AAV injection. As shown in FIG. 16A-16C, CD3 staining was observed in the AAV injected hippocampus especially clustering near the ventricle. Both Aβ plaques and CD3+ T cells were observed in the hippocampus of mice two months post-AAV injection. See FIG. 17A-17C. Loss synaptic markers in areas covered by amyloid plaques in the hippocampus was also observed, as shown in FIG. 18A-18C.

### Example 2. Aβ Plaques Induce NP-Tau and Enhance p-Tau in P301S Tauopathy Mouse Model

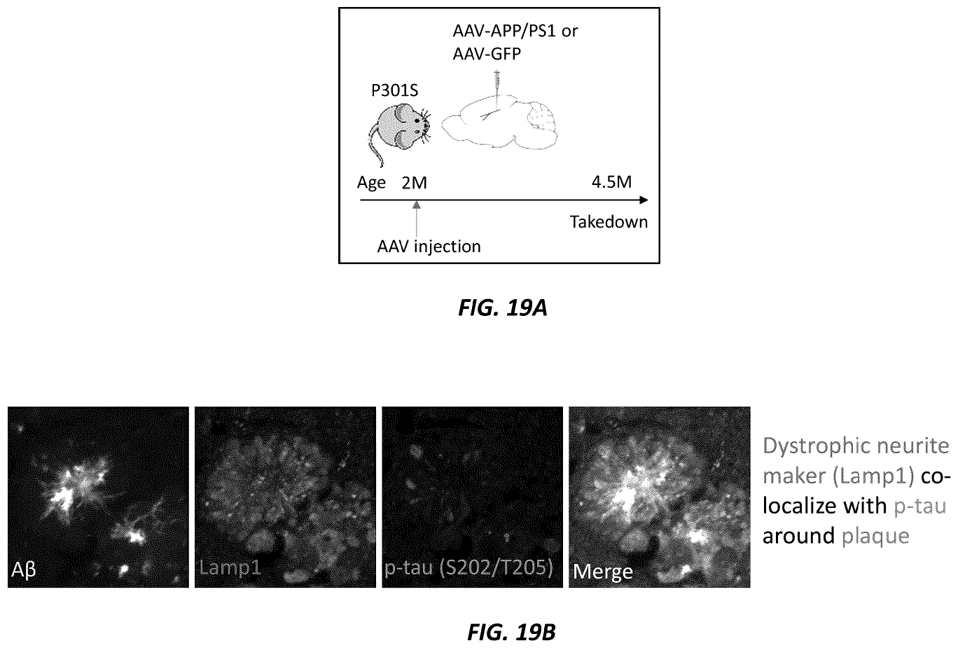

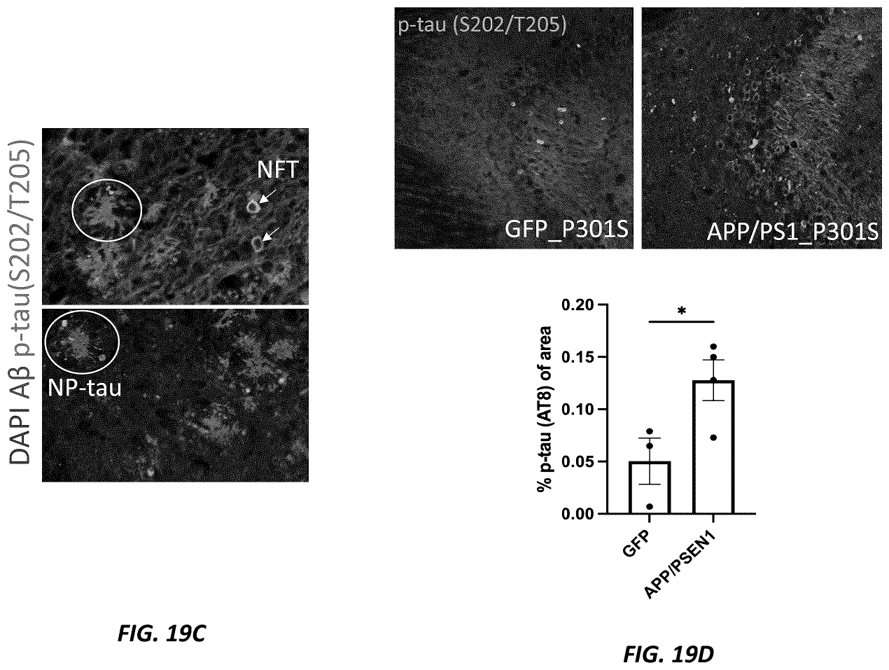

Existing tau mouse models are lacking amyloid plaque-associated phosphorylated tau pathology, which is a prominent phenotype in Alzheimer's disease. To generate a mouse model with both amyloid plaque and tau pathology, which are the hallmarks of Alzheimer's disease, we injected the AAV vectors carrying APP and PSEN1 with the mutations described in Example 1 into the hippocampus of the tau P301S mouse model (PS19 mice; available at jax.org/strain/008169, herein incorporated by reference in its entirety for all purposes) (FIG. 19A). To compare the severity of tau pathology, we injected AAV-GFP as control. We took down the mice 2.5 months post-AAV injection to examine amyloid and tau pathology. Mice injected with AAV-APP/PS1 exhibit robust amyloid plaque pathology and the presence of plaque-associated phosphorylated tau pathology, which is located in the peri-plaque region and co-localized with dystrophic neurite marker Lamp1 (FIG. 19B). This is the so-called neuritic plaque-tau pathology (NP-tau). We also observed the classical phosphorylated-tau accumulation in the cytoplasm, known as neurofibrillary tangles (NFT) (FIG. 19C). When comparing the phosphorylated-tau signals in mice injected with AAV-APP/PS1 versus AAV-GFP, we found that mice injected with AAV-APP/PS1 exhibited higher phosphorylated-tau signals (FIG. 19D), suggesting that amyloid plaques promote tau pathology. In summary, we showed that AAV-APP/PS1 injection in the tau P301S mice induced amyloid plaque formation and presence of the NP-tau pathology. Such a model will be useful in understanding how therapeutics modify amyloid plaque induced tau pathology.

### Example 3. Prophylactic Aβ Antibody (Aducanumab [mIgG2a]) Treatment Reduces Amyloid Plaques and Dystrophic Neurites

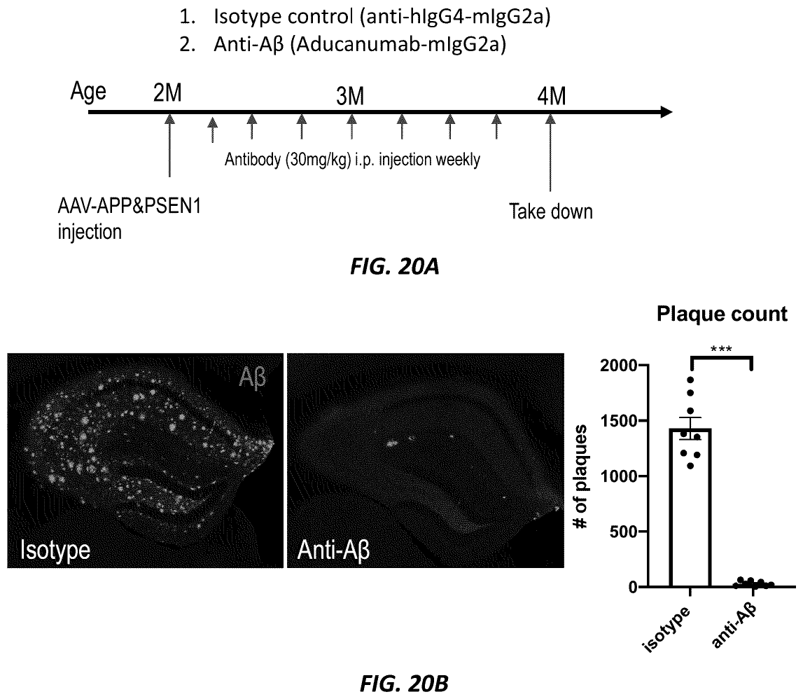

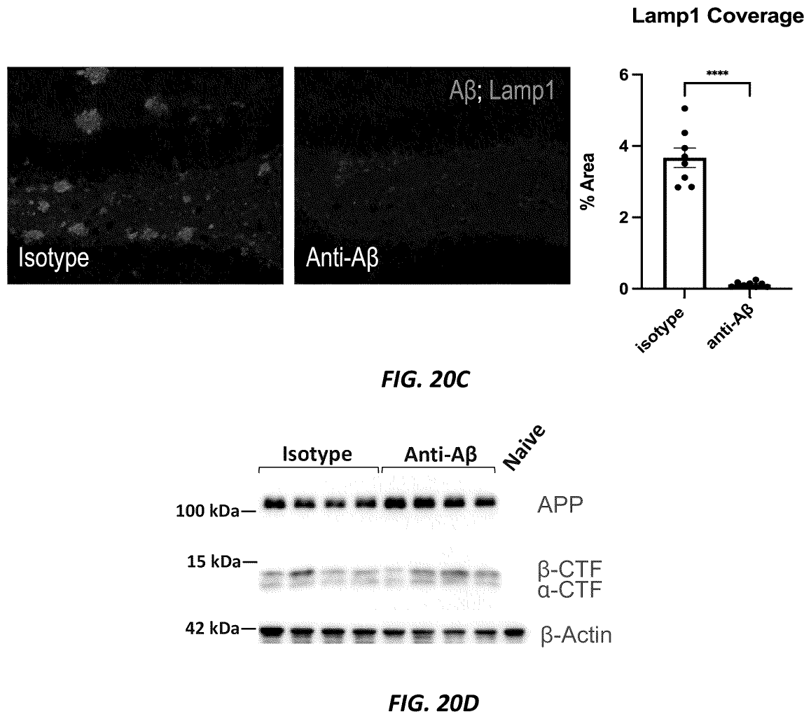

We tested an anti-Aβ antibody, Aducanumab, which is known to clear plaques in Alzheimer's patients, in the AAV-APP/PS1 mouse model described in Example 1. We started antibody treatment 1 week post-AAV injection in the hippocampus to induce amyloid plaque deposition. We did weekly antibody administration for 7 weeks and took down the mice 1 week after the final antibody administration (FIG. 20A). We found that Aducanumab treatment was able to significantly reduce plaque levels compared to mice treated with control antibody (FIG. 20B). Aducanumab treatment also significantly reduced plaque-associated dystrophic neurite marker Lamp1 (FIG. 20C). It did not change the levels of APP and the intracellular APP C-terminal fragments (FIG. 20D), suggesting that anti-Aβ antibody mainly clears the extracellular plaques. This demonstrates that our model can be used to test Alzheimer's disease therapeutics.

### Example 4. Rapid Accumulation of Aβ Plaques can be Induced in Various Mouse Models, Including Mice Humanized for APOE, to Model AD Pathology and Test Different Therapeutic Approaches

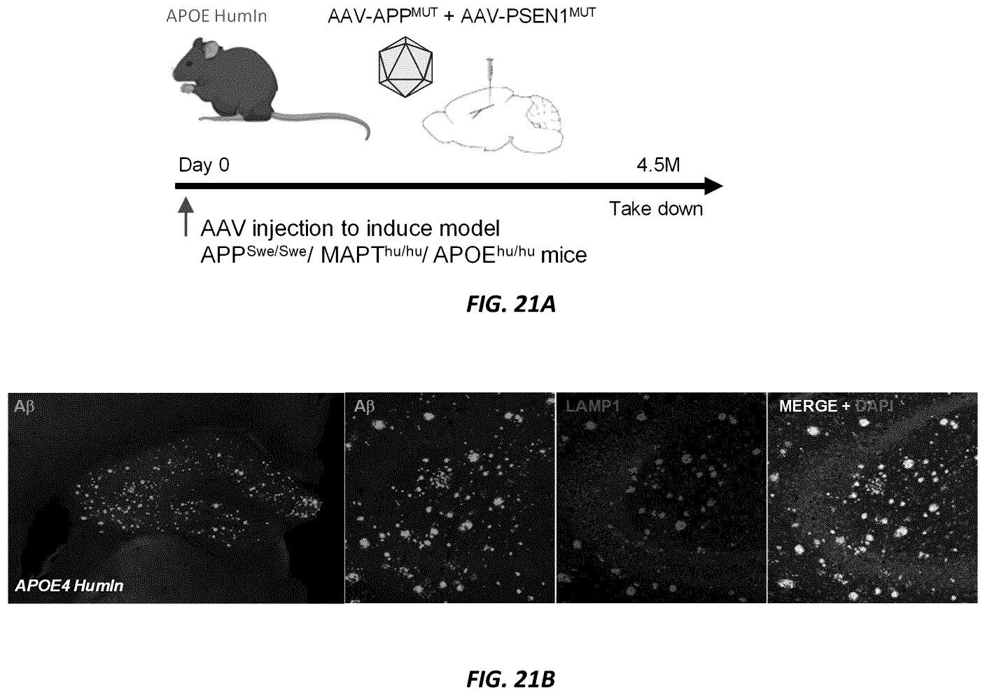

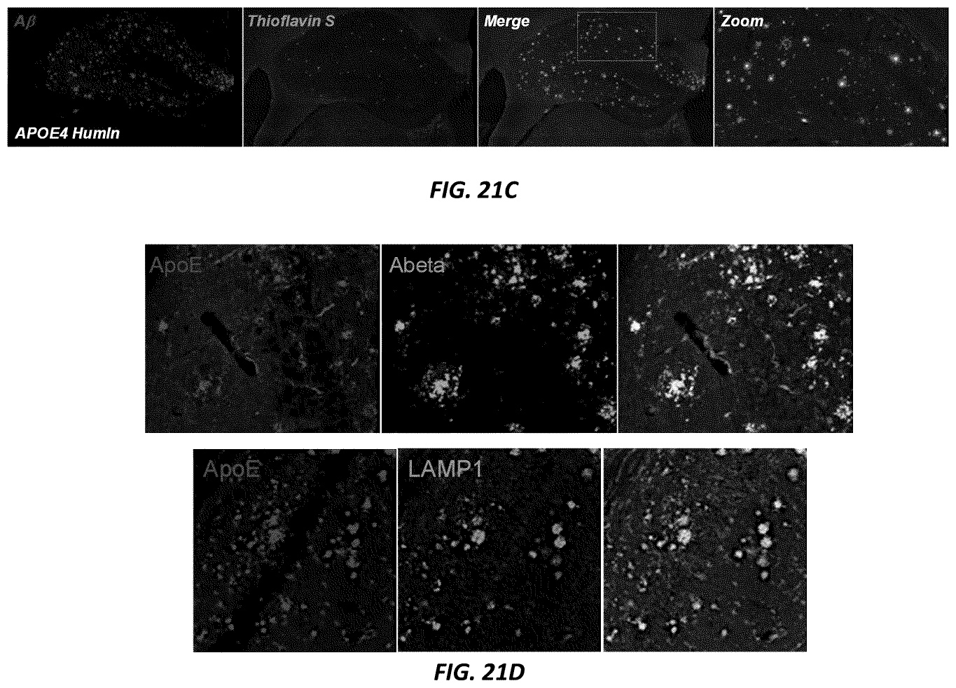

An advantage of the AAV-mediated rapid amyloid plaque model described in Example 1 is the ability to induce amyloid-β pathology in various mouse models to characterize the interplay of different human genes and test additional therapeutics for the treatment of amyloid disorders. These may include applying this AAV-driven model to mice expressing other humanized genes associated with Alzheimer's disease (AD) to investigate mechanisms related to disease pathogenesis in the context of additional human genetic risk factors associated with increased disease risk, progression or severity. For example, APOE polymorphic alleles are the major genetic determinant of late-onset AD risk and onset. Of the three common APOE haplotypes, the APOE e4 (APOE4) allele confers a substantially increased risk of developing AD, while the APOE e2 (APOE2) allele confers a an exceptionally low likelihood of developing the disease, compared to the most common allele, APOE e3 (APOE3). We tested the application of this AAV-driven model in mice expressing human APOE via in-locus, targeted replacement of murine APOE with human APOE4 (FIG. 21A). We observed that amyloid plaque pathology can be induced in these humanized mice, with substantial amyloid accumulation observed within months of AAV injection (FIGS. 21B-21D). We also found that these mice accumulate additional features of amyloid-associated pathology, including the co-deposition of human ApoE protein in Aβ plaques (FIG. 21D), and the presence of plaque-associated neuritic dystrophy, an indication of neuronal damage (FIG. 21B). The ability of this approach to induce multiple AD-associated pathologies in additional humanized mouse models provides an example for the utility of this AAV-driven approach to further study and characterize the function of additional genes of interest, and the ability to test therapeutics targeting these genes for the treatment of amyloidogenic disease such as AD.

1. A composition comprising one or more vectors comprising a nucleic acid encoding amyloid-beta precursor protein and a nucleic acid encoding presenilin-1.

2.-36. (canceled)

37. A cell comprising the composition of claim 1.

38.-47. (canceled)

48. A non-human animal comprising the composition of claim 1.

49.-61. (canceled)

62. A method of modeling Alzheimer's disease in a non-human animal, comprising administering the composition of claim 1 to the non-human animal.

63. The method of claim 62, wherein the non-human animal is a mammal.

64. The method of claim 62, wherein the non-human animal is a non-human primate or a rodent.

65. (canceled)

66. The method of claim 62, wherein the non-human animal is a mouse.

67. A method of making the non-human animal of claim 48, comprising administering the composition to a non-human animal.

68. The method of claim 62, wherein the composition is administered to the brain of the non-human animal.

69. The method of claim 62, wherein the composition is administered to the non-human animal via intrahippocampal injection, intracerebroventricular injection, intracranial injection, intrathecal injection, or stereotactic injection.

70. The method of claim 62, wherein:
(I) the one or more vectors comprise a first vector comprising the nucleic acid encoding the amyloid-beta precursor protein and a second vector comprising the nucleic acid encoding the presenilin-1, and wherein the first and second vectors are administered simultaneously; or
(II) the one or more vectors comprise a first vector comprising the nucleic acid encoding the amyloid-beta precursor protein and a second vector comprising the nucleic acid encoding the presenilin-1, and wherein the first and second vectors are administered separately or sequentially.

71. (canceled)

72. The method of claim 62, wherein the non-human animal comprises a human microtubule-associated protein tau coding sequence comprising a tauopathy-associated mutation, wherein the microtubule-associated protein tau is expressed, wherein the human microtubule-associated protein tau coding sequence is genomically integrated, and wherein the tauopathy-associated mutation is a P301S mutation.

73. The method of claim 62, wherein:
(I) the non-human animal comprises a humanized APOE genomic locus, wherein a human apolipoprotein E4 protein is expressed from the humanized APOE genomic locus;
(II) the non-human animal comprises a humanized MAPT genomic locus, wherein a human microtubule-associated protein tau protein is expressed from the humanized MAPT genomic locus;
(III) the non-human animal comprises a humanized APP genomic locus, wherein a chimeric non-human animal/human amyloid-beta precursor protein comprising K670N/M671L mutations with reference to a human APP770 isoform or K595N/M596L mutations with reference to a human APP695 isoform is expressed from the humanized APP genomic locus; or
(IV) any combination thereof.

74. The method of claim 73, wherein the non-human animal comprises the humanized APOE genomic locus, the humanized MAPT genomic locus, and the humanized APP genomic locus.

75. The method of claim 62, wherein the non-human animal develops one or more Alzheimer's disease phenotypes after administration of the composition.

76. The method of claim 62, wherein the non-human animal develops:
(I) one or more or all of the following after administration of the composition compared to a control non-human animal that has not been administered the composition: Aβ plaque pathology; loss of synapses; gliosis; and dystrophic neurites around Aβ plaques;
(II) one or more or all of the following after administration of the composition compared to a control non-human animal that has not been administered the composition: tau aggregates in dystrophic neurites surrounding Aβ plaques (NP-tau); neurofibrillary tangles (NFTs); and increased phosphorylated tau in the somatodendritic compartment of neurons; or
(III) the following after administration of the composition compared to a control non-human animal that has not been administered the composition: co-deposition of ApoE protein in Aβ plaques.

77. (canceled)

78. (canceled)

79. The method of claim 75, wherein the non-human animal develops one or more Alzheimer's disease phenotypes within two months or one month after administration of the composition.

80. (canceled)

81. The method of claim 75, wherein the non-human animal develops Aβ plaque pathology within one month after administration of the composition.

82. (canceled)

83. A method of assessing a therapeutic candidate for the treatment of Alzheimer's disease, comprising:
(a) administering a candidate agent to the non-human animal of claim 48;
(b) performing one or more assays to determine if the candidate agent has an effect on an Alzheimer's disease phenotype; and
(c) identifying the candidate agent that ameliorates the Alzheimer's disease phenotype as a therapeutic candidate.

84. (canceled)

85. A method of assessing a therapeutic candidate for the prevention of Alzheimer's disease, comprising:
(a) administering a candidate agent to a non-human animal;
(b) administering the composition of claim 1 to the non-human animal;
(c) performing one or more assays to determine if the candidate agent has an effect on an Alzheimer's disease phenotype; and
(d) identifying the candidate agent that prevents the Alzheimer's disease phenotype as a therapeutic candidate.

86.-101. (canceled)

102. The method of claim 62, wherein the amyloid-beta precursor protein is an APP695 amyloid-beta precursor protein isoform.

103. The method of claim 62, wherein the amyloid-beta precursor protein comprises one or more mutations associated with familial Alzheimer's disease.

104. The method of claim 62, wherein the amyloid-beta precursor protein comprises three mutations associated with familial Alzheimer's disease.

105. The method of claim 62, wherein the amyloid-beta precursor protein comprises K670N/M671L, E693G, and I716F mutations with reference to a human APP770 isoform or K595N/M596L, E618G, and I641F mutations with reference to a human APP695 isoform.

106. The method of claim 62, wherein the amyloid-beta precursor protein is at least 90%, at least 95%, at least 96%, at least 97%, at least 98%, or at least 99% identical to the sequence set forth in SEQ ID NO: 1, or wherein the nucleic acid encoding the amyloid-beta precursor protein is at least 90%, at least 95%, at least 96%, at least 97%, at least 98%, or at least 99% identical to the sequence set forth in SEQ ID NO: 2.

107. The method of claim 62, wherein the amyloid-beta precursor protein comprises the sequence set forth in SEQ ID NO: 1, or wherein the nucleic acid encoding the amyloid-beta precursor protein comprises the sequence set forth in SEQ ID NO: 2.

108. The method of claim 62, wherein the presenilin-1 comprises one or more mutations associated with familial Alzheimer's disease.

109. The method of claim 62, wherein the presenilin-1 comprises two mutations associated with familial Alzheimer's disease.

110. The method of claim 62, wherein the presenilin-1 comprises the following mutations: M146L and L286V.

111. The method of claim 62, wherein the presenilin-1 is at least 90%, at least 95%, at least 96%, at least 97%, at least 98%, or at least 99% identical to the sequence set forth in SEQ ID NO: 3, or wherein the nucleic acid encoding the presenilin-1 is at least 90%, at least 95%, at least 96%, at least 97%, at least 98%, or at least 99% identical to the sequence set forth in SEQ ID NO: 4.

112. The method of claim 62, wherein the presenilin-1 comprises the sequence set forth in SEQ ID NO: 3, or wherein the nucleic acid encoding the presenilin-1 comprises the sequence set forth in SEQ ID NO: 4.

113. The method of claim 62, wherein the amyloid-beta precursor protein comprises K670N/M671L, E693G, and I716F mutations with reference to a human APP770 isoform or K595N/M596L, E618G, and I641F mutations with reference to a human APP695 isoform, and
wherein the presenilin-1 comprises the following mutations: M146L and L286V.

114. The method of claim 62, wherein:
(I) the amyloid-beta precursor protein is at least 90%, at least 95%, at least 96%, at least 97%, at least 98%, or at least 99% identical to the sequence set forth in SEQ ID NO: 1, and
the presenilin-1 is at least 90%, at least 95%, at least 96%, at least 97%, at least 98%, or at least 99% identical to the sequence set forth in SEQ ID NO: 3; or
(II) the nucleic acid encoding the amyloid-beta precursor protein is at least 90%, at least 95%, at least 96%, at least 97%, at least 98%, or at least 99% identical to the sequence set forth in SEQ ID NO: 2, and
the nucleic acid encoding the presenilin-1 is at least 90%, at least 95%, at least 96%, at least 97%, at least 98%, or at least 99% identical to the sequence set forth in SEQ ID NO: 4.

115. The method of claim 62, wherein:
(I) the amyloid-beta precursor protein comprises the sequence set forth in SEQ ID NO: 1, and
the presenilin-1 comprises the sequence set forth in SEQ ID NO: 3; or
(II) the nucleic acid encoding the amyloid-beta precursor protein comprises the sequence set forth in SEQ ID NO: 2, and
the nucleic acid encoding the presenilin-1 comprises the sequence set forth in SEQ ID NO: 4.

116. The method of claim 62, wherein the nucleic acid encoding the amyloid beta precursor protein is operably linked to a constitutive promoter, a tissue-specific promoter, or an inducible promoter.

117. The method of claim 62, wherein the nucleic acid encoding the amyloid beta precursor protein is operably linked to a neuron-specific promoter, a synapsin-1 promoter, or a human synapsin-1 promoter.

118. The method of claim 62, wherein the nucleic acid encoding the presenilin-1 is operably linked to a constitutive promoter, a tissue-specific promoter, or an inducible promoter.

119. The method of claim 62, wherein the nucleic acid encoding the presenilin-1 is operably linked to a neuron-specific promoter, a synapsin-1 promoter, or a human synapsin-1 promoter.

120. The method of claim 62, wherein the nucleic acid encoding the amyloid beta precursor protein is operably linked to a constitutive promoter, a tissue-specific promoter, or an inducible promoter, and
wherein the nucleic acid encoding the presenilin-1 is operably linked to a constitutive promoter, a tissue-specific promoter, or an inducible promoter.

121. The method of claim 62, wherein the nucleic acid encoding the amyloid beta precursor protein is operably linked to a neuron-specific promoter, a synapsin-1 promoter, or a human synapsin-1 promoter, and
wherein the nucleic acid encoding the presenilin-1 is operably linked to a neuron-specific promoter, a synapsin-1 promoter, or a human synapsin-1 promoter.

122. The method of claim 62, wherein the one or more vectors comprise one or more viral vectors.

123. The method of claim 62, wherein the one or more vectors comprise one or more adeno-associated virus (AAV) vectors.

124. The composition of claim 123, wherein the one or more AAV vectors comprise one or more recombinant AAV9 vectors.

125. The method of claim 62, wherein the amyloid-beta precursor protein comprises K670N/M671L, E693G, and I716F mutations with reference to a human APP770 isoform or K595N/M596L, E618G, and I641F mutations with reference to a human APP695 isoform or wherein the amyloid-beta precursor protein comprises the sequence set forth in SEQ ID NO: 1,
wherein the presenilin-1 comprises the following mutations: M146L and L286V or wherein the presenilin-1 comprises the sequence set forth in SEQ ID NO: 3,
wherein the nucleic acid encoding the amyloid beta precursor protein is operably linked to a neuron-specific promoter or wherein the promoter is a human synapsin-1 promoter, and
wherein the nucleic acid encoding the presenilin-1 is operably linked to a neuron-specific promoter or wherein the promoter is a human synapsin-1 promoter, and
wherein the one or more vectors comprise one or more adeno-associated virus (AAV) vectors or one or more recombinant AAV9 vectors.

126. The method of claim 62, wherein the one or more vectors comprise a first vector comprising the nucleic acid encoding the amyloid-beta precursor protein and a second vector comprising the nucleic acid encoding the presenilin-1.

127. The method of claim 62, wherein the one or more vectors comprise a single vector comprising the nucleic acid encoding the amyloid-beta precursor protein and the nucleic acid encoding the presenilin-1.


SEQUENCE LISTING (11 sequences)

SEQ ID NO: 1 - protein, 695 aa, synthetic construct
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAGDVGSNKGAIIGLMVGGVVIATVFVITLVMLKKKQYTSIHHGVVEVDAAVTPEERHLSKMQQNGYENPTYKFFEQMQN

SEQ ID NO: 2 - DNA, 2088 bp, synthetic construct
  source 1..2088: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatttggatgcagaattccgacatgactcaggatatgaagttcatcatcaaaaattggtgttctttgcaggagatgtgggttcaaacaaaggtgcaatcattggactcatggtgggcggtgttgtcatagcgacagtgttcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 3 - protein, 467 aa, synthetic construct [sequence omitted]

SEQ ID NO: 4 - DNA, 1404 bp, synthetic construct
  source 1..1404: mol_type = other DNA
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcctgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctgtcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 5 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - DNA, 2088 bp, Homo sapiens
  source 1..2088: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 7 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 8 - DNA, 2313 bp, Homo sapiens
  source 1..2313: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 9 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 10 - DNA, 1404 bp, Homo sapiens
  source 1..1404: mol_type = other DNA
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 11 - protein, 48 aa, Homo sapiens
KMDAEFRHDSGYEVHHQKLVFFAEDVGSNKGAIIGLMVGGVVIATVIV